(19) United States
(12) Patent Application Publication
(10) Pub. No.: US 2025/0283019 A1
(43) Pub. Date: Sep. 11, 2025

(54) NEURO-INTEGRATED BIOREACTOR SYSTEM FOR STUDYING JOINT PAIN AND TREATMENT THEREOF

(71) Applicant: University of Pittsburgh - Of the Commonwealth System of Higher Education, Pittsburgh, PA (US)

(72) Inventors: Hang Lin, Pittsburgh, PA (US); Michael S. Gold, Oakmont, PA (US); Rocky Sung Chi Tuan, Pittsburgh, PA (US)

(73) Assignee: University of Pittsburgh - Of the Commonwealth System of Higher Education, Pittsburgh, PA (US)

(21) Appl. No.: 18/859,083

(22) PCT Filed: May 1, 2023
(86) PCT No.: PCT/US2023/020620

§ 371 Date: Oct. 22, 2024

Related U.S. Application Data
(60) Provisional application No. 63/337,053, filed on Apr. 30, 2022.

(51) Int. Cl. C12M 3/00; C12M 1/00; C12M 1/12; C12M 1/34; C12M 1/42; C12N 5/071; C12N 5/077; C12N 5/0786; C12N 5/0793; G01N 33/50

(52) U.S. Cl. CPC C12M 21/08; C12M 23/22; C12M 23/34; C12M 23/40; C12M 25/02; C12M 25/14; C12M 29/10; C12M 35/02; C12M 41/46; C12N 5/0619; C12N 5/0645; C12N 5/0653; C12N 5/0654; C12N 5/0655; C12N 5/0697; G01N 33/5091

(57) ABSTRACT

Disclosed herein are various bioreactor devices that mimic the mammalian joint. The bioreactor device includes a series of bioreactor chambers that contain different components of the joint, such as bone, cartilage, synovium, and ligament, and which are integrated with neural processes to better recapitulate physiological conditions, including joint pain. At least two different nutrient fluid circulation systems connect subsets of the bioreactor chambers to differentially supply nutrient fluids at concentrations optimized for the tissue that the fluid nourishes. The disclosed bioreactor devices enable interrogation of the interplay between the peripheral nervous system and joint tissues. By recording activity in sensory neurons, joint integrity as well as therapeutic efficacy can be monitored in real time.

### ACKNOWLEDGMENT OF GOVERNMENT SUPPORT

This invention was made with government support under grant number TR003090 awarded by the National Institutes of Health. The government has certain rights in the invention.

### FIELD

The present disclosure relates to the engineering of a three-dimensional human micro-joint chip, physiologically analogous to the native joint and capable of modeling pathogenesis and treatment of joint diseases for the screening and development of disease-modifying treatments such as medications.

### BACKGROUND

Life's wear and tear can leave joints damaged. All too often, the result is joint pain, such as the pain and disability of osteoarthritis (OA). Because pain is the most debilitating symptom of OA, it remains the primary target for therapeutic interventions. However, the mechanism underlying OA pain has not been fully understood. In addition, there is no long-term, safe and efficacious medication to manage OA pain. Limitations of current in vitro cell culture and laboratory animal models pose additional barriers to the successful development of joint pain medications, including for OA pain.

Therefore, there is a need in the art for a physiologically relevant in vitro model that includes all components of the joint, such as the cartilage, bone, synovium, infrapatellar fat pad (IPFP), and sensory neurons, and retains the heterogeneity of the various tissues within the joint, to provide a model of the joint, and for use in developing more effective or and personalized therapies for diseases of the joint.

### SUMMARY

Disclosed herein is a joint-on-a-chip tissue bioreactor (neu-microJoint), that integrates an engineered osteochondral complex, synovium, adipose tissue, and nerve cells, enabling interrogation of the dynamic interplay between the peripheral nervous system and joint tissues. By recording activity in sensory neurons, joint integrity as well as therapeutic efficacy can be monitored in real time.

In some examples, the neu-microJoint replicates known stratifications and physiologic conditions in human OA, inflamed arthritis and diabetic-induced complications of diabetes and other joint diseases (for example in the knee) to study and mimic the cause of onset, effect on target tissue elements and disease progression, including pain. Thus, the neu-microJoint can be used to model a mammalian joint, such as a human joint, and pain within the joint.

In some examples, a bioreactor is provided, comprising four chambers as follows:
i) a first chamber comprising an upper part and a lower part, wherein the upper part of the first chamber comprises a chondrocytes within a tissue scaffold, and the lower part of the first chamber comprises a osteoblasts within a tissue scaffold;
ii) a second chamber comprising an upper part and a lower part, each comprising synovial cells within a tissue scaffold;
iii) a third chamber comprising an upper part and a lower part, each comprising adipose cells within a tissue scaffold; and
iv) a fourth chamber comprising sensory neurons in a two-dimensional culture, wherein the fourth chamber is interconnected to the lower parts of the first, second, and third chambers by microchannels.

The bioreactor further comprises a first influx conduit that supplies a first nutrient fluid to the lower part of the first chamber, and a first efflux conduit that removes the first nutrient fluid from the lower part of the first chamber; a second influx conduit that supplies a second nutrient fluid to the lower part of the second chamber, and a second efflux conduit that removes the second nutrient fluid from the lower part of the second chamber; a third influx conduit that supplies a third nutrient fluid to the lower part of the third chamber, and a third efflux conduit that removes the third nutrient fluid from the lower part of the third chamber; and a fourth influx conduit that supplies a fourth nutrient fluid to the upper parts of the first, second, and third chambers, and a fourth efflux conduit that removes the fourth nutrient fluid from the upper parts of the first, second, and third chambers.

The microchannels of the bioreactor are configured to allow growth of neurites of the sensory neurons from the fourth chamber to the lower parts of the first, second, and third chambers, and to limit bulk flow of the first, second, third, and fourth nutrient fluids into the fourth chamber. The upper parts of the first, second, and third chambers are interconnected via fluid conduits in series. The upper and lower parts of the first, second and third chambers are separated by a barrier layer that permits biochemical communication but not cell migration between the upper and lower parts of the first, second and third chambers, respectively. The chondrocyte tissue scaffold is exposed to the fourth nutrient fluid and not the first, second, or third nutrient fluids. The osteoblast tissue scaffold is exposed to the first nutrient fluid and not the second, third, or fourth nutrient fluids.

The bioreactor comprises a perturbation source that provides a preselected perturbation to at least one of the first, second, third, or fourth chambers. In some examples, the perturbation source is an agent that is included in the nutrient fluid circulating in the bioreactor.

Methods of using the disclosed neu-microJoint bioreactor, such as to model a mammalian joint, for example a human joint, are disclosed, as are methods of testing an agent for modification of biological function (e.g., pain sensation) in a mammalian joint.

The foregoing and other objects, features, and advantages of the invention will become more apparent from the following detailed description, which proceeds with reference to the accompanying figures.

### BRIEF DESCRIPTION OF THE DRAWINGS

FIG. 1 is a schematic diagram illustrating the cellular organization of a bioreactor system for studying joint pain. Individual chambers containing adipose, synovium, and cartilage/bone cells are innervated with sensory neurons. The cell bodies of the sensory neurons are located in a central chamber. Neurites of the sensory neurons travel through microchannels to outer chambers containing the adipose, synovium and cartilage/bone tissue. Neurites also travel via microchannels to the adipose and bone chambers, not shown.

FIG. 2A shows a three-dimensional rendering of an exemplary neu-microJoint bioreactor, illustrating the cell/tissue chambers and conduits for nutrient flow.

FIG. 2B shows a three-dimensional rendering of an exemplary neu-microJoint bioreactor, illustrating the upper and lower parts of the tissue chambers, which are separated by a barrier, such as an o-ring and semi-permeable barrier.

FIG. 2C shows a three-dimensional rendering of an example of an exemplary neu-microJoint bioreactor, illustrating the directional flow of nutrient fluid. Hollow arrows indicate flow of nutrient fluid to the lower parts of individual tissue chambers. Shaded arrows indicate the flow of nutrient fluid through the upper parts of the three tissue chambers in series.

FIG. 3 shows a three-dimensional rendering of an example of an exemplary neu-microJoint bioreactor, illustrating details of the bioreactor.

FIG. 4 shows a plan view of the shell of an exemplary neu-microJoint bioreactor.

FIG. 5 is a photograph showing a perspective view of the shell of an exemplary neu-microJoint bioreactor, illustrating an example of construction for the shell.

FIG. 6 is a photograph showing a top view of the shell of an exemplary neu-microJoint bioreactor.

FIG. 7 shows a three-dimensional renderings of an exemplary inner body and ring for use in the disclosed bioreactor system, from two different views.

FIG. 8 shows a three-dimensional rendering of the components of an exemplary bioreactor, in an exploded view.

FIGS. 9A-9D show three-dimensional renderings of exemplary inner bodies for use in bioreactor systems, from two different views.

FIG. 10 shows a schematic, cross-sectional side view of an exemplary bioreactor chamber having a plural different tissue types growing therein.

FIG. 11 is a digital image showing calcium imaging of neurons in an exemplary neu-microJoint bioreactor, illustrating growth of neurites from DRG cell bodies in the neuron chamber through microchannels to an adjacent tissue chamber.

FIG. 12 is a graph showing recording of action potentials of DRG neurons in an exemplary neu-microJoint bioreactor, elicited in response to axonal stimulation. The neural signals were recorded using an electrode microarray located in the bottom of the neural chamber of the neu-microJoint bioreactor.

FIG. 13 is a digital image showing calcium imaging of neurons in an exemplary neu-microJoint bioreactor, illustrating growth of neurites from DRG cell bodies in the neuron chamber through microchannels to an adjacent tissue chamber containing 3D gel tissue construct of MSCs.

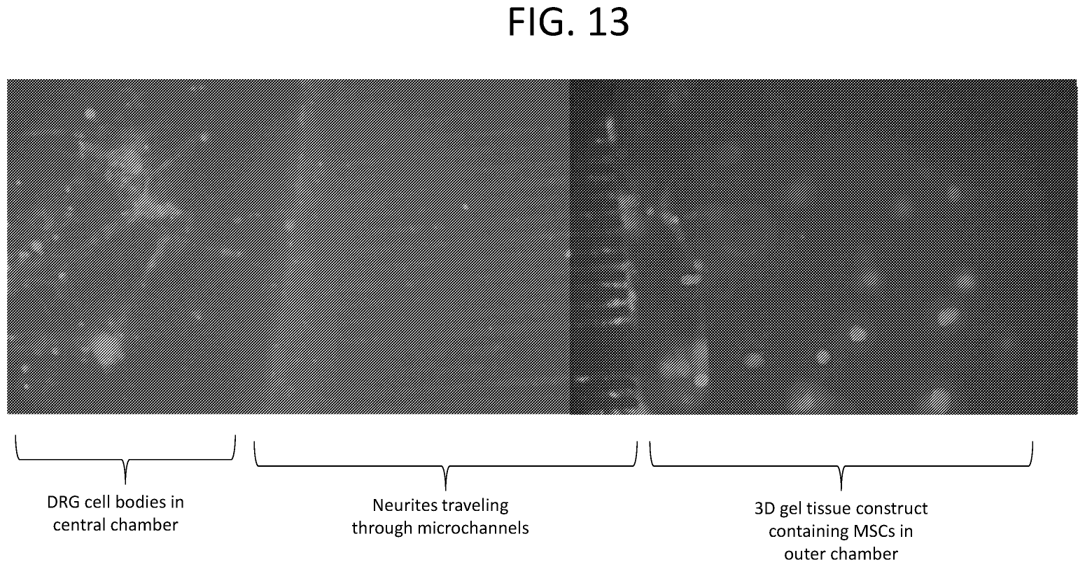

FIG. 14. Measured responses of sensory neurons in the neu-microJoint bioreactor to different agents applied to the synovium-specific fluid channel. A subpopulation of neurons respond to OA-modeled synovium fluid.

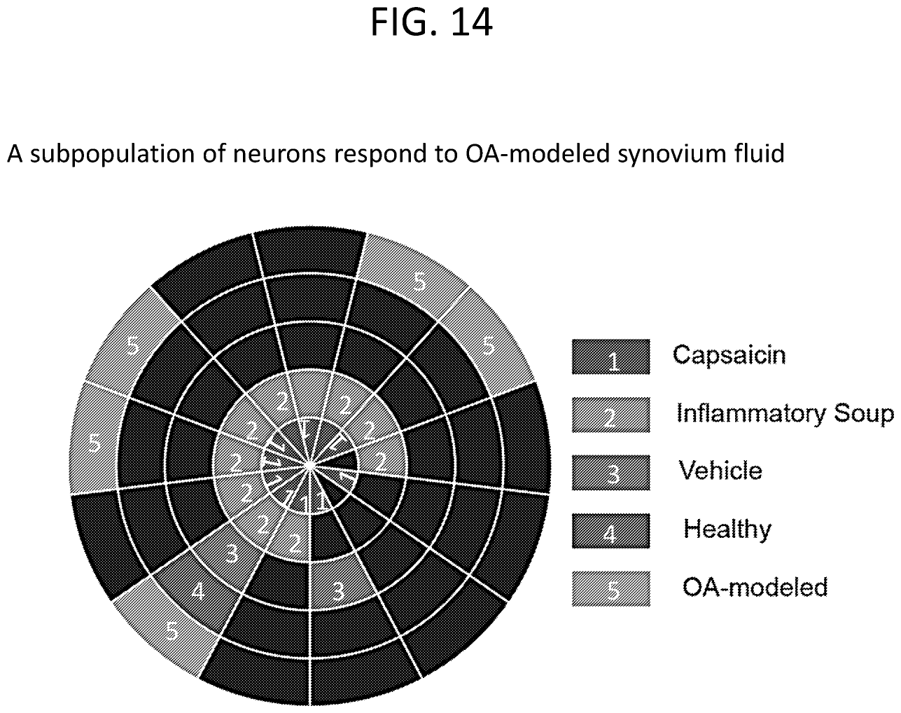

### DETAILED DESCRIPTION

Disclosed herein is a joint-on-a-chip tissue bioreactor (neu-microJoint), that integrates engineered osteochondral complex, synovium, adipose tissue, and sensory nerve connections, enabling interrogation of the dynamic interplay between the peripheral nervous system and joint tissues. In the neu-microJoint bioreactor, major joint elements, such as bone, cartilage, synovium and adipose tissue, are typically engineered from human cells. In particular, synovium is innervated with neurons, allowing the assessment of pain level. The neu-microJoint bioreactor represents a robust platform for joint pain research and therapeutic drug screening.

In some examples, the neu-microJoint bioreactor models known stratifications and physiologic conditions in human OA, inflamed arthritis and diabetic-induced complications of diabetes and other joint diseases (for example in the knee) to study and mimic the cause of onset, effect on target tissue elements and disease progression. In further examples, the tissue bioreactor models a whole joint, and is suitable for the initiation or acceleration of joint diseases with different pathophysiologic mechanisms to allow the investigation of disease onset and progression, the development of therapeutics that target different tissues and/or pathways, and the assessment of direct and indirect effects of candidate drugs.

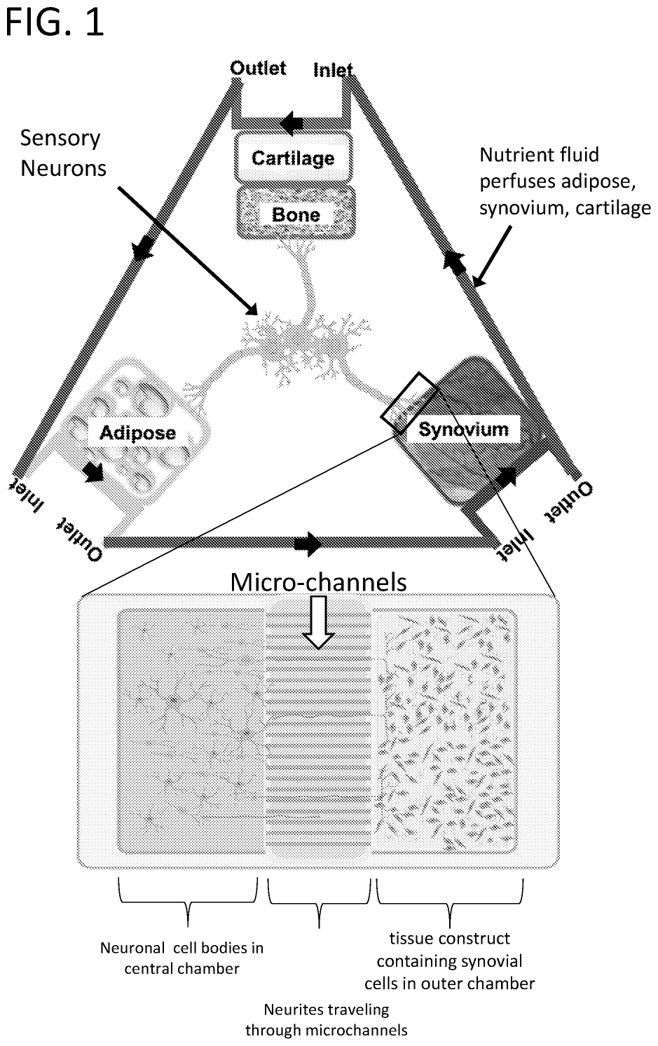

Referring to FIGS. 1-3, to study mechanisms of joint-associated pain (for example, due to OA), develop novel treatments, screen novel treatments, and identify the most effective treatment option for individual patients (personalized pain medicine), the neu-microJoint bioreactor contains tissue scaffolds, in which bone, cartilage, synovium, and adipose-like tissues are integrated. This tissue is housed in the outer chambers of the neu-microJoint, and in a three-dimensional context. In the center of the neu-microJoint is a smaller chamber, which is used to house sensory neurons in a standard 2D culture. These neural cells innervate the joint tissues in the outer chambers via microfluidic channels travelling from the central chamber to the outer chambers. The microfluidic channels take advantage of surface tension and fluid resistance to enable a physical connection between the neural chamber and the tissue chambers in the neu-microJoint bioreactor, while restricting bulk fluid exchange. Thus, it is possible to maintain the phenotype of each tissue type with tissue specific culture media, while enabling innervation of the tissue. Because the neural cells are cultured in 2D (e.g., on a glass surface), it is possible to monitor neural activity using standard imaging and/or electrophysiological methods.

In vivo, the cell body of sensory neurons that innervate joint tissues reside in dorsal root ganglia (DRG), located adjacent to the spinal cord and consequently a considerable distance from the joint. This orientation is recapitulated in the disclosed neu-microJoint bioreactor, with the sensory neurons cell bodies maintained in isolation from the joint tissues. The only connections between the neurons and the tissues is provided by the neurites. Consequently, activity detected in the cell body is evoked by mediators released from the tissues and cells in the outer chambers that are able to generate action potentials. Given the differential localization within a sensory neuron between the cell body and terminals, the disclosed neu-microJoint bioreactor is a more anatomically accurate structure than prior models of joint pain.

An advantage of separating the tissues but allowing cross-talk as well as the independent innervation of all three allowing tissues is that it is possible to determine the tissue(s) responsible for the pain of OA, whether there are tissues that release compounds that attenuate the pain of OA, and how the response of any one tissue type influences the phenotype of the others (and is therefore a primary or secondary driver for OA pain).

Further, in the disclosed neu-microJoint bioreactor the neurites physically connect with joint tissues, which allows studying the direct contribution of mechanics on neurites within tissues. The 2D (neural culture)/3D (joint tissues) aspect of the neu-microJoint bioreactor enables assessment of changes in neural activity with imaging and/or microelectrode arrays with innervation of tissue grown in 3D. Advantages of the 3D tissues include 1) more accurate representation of tissue function and phenotype; and 2) the ability to apply mechanical (and therefore more physiologically relevant) stimuli.

### I. Summary of Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. The term “comprises” means “includes.” Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various aspects, the following explanations of terms are provided:

Bioreactor: Any manufactured or engineered device or system that supports a biologically active environment. In one example, a bioreactor includes reactor chambers which are a set of vessels in which a chemical process is carried out which involves organisms or biochemically active substances derived from such organisms. A bioreactor may also include a device or system meant to grow cells or tissues in the context of cell culture for use in tissue engineering or biochemical engineering. On the basis of mode of operation, a bioreactor may be classified as batch, fed batch or continuous (e.g. a continuous stirred-tank reactor model). Cells growing in bioreactors may be submerged in liquid medium in suspension or may be attached to the surface of a solid medium. Suspension bioreactors can use a wider variety of organisms and cells, since special attachment surfaces are not needed, and can operate at much larger scale than immobilized cultures. However, in a continuously operated process the cells will be removed from the reactor with the effluent. Immobilization is a general term describing a wide variety of cell or particle attachment or entrapment. Immobilization is useful for continuously operated processes, since the cells will not be removed with the reactor effluent, but can be more limited in scale (for example, cell number) because the cells are only present on the surfaces of the vessel.

A perfusion bioreactor is a bioreactor that includes one or more reactor chambers that have an inlet and outlet that provide for the provision of medium and the removal of waste or spent medium from the bioreactor at a specified flow rate. In a perfusion bioreactor for the cultivation of mammalian cells, medium is perfused through the bioreactor at a specified rate while the cell mass is contained within the bioreactor by means of a cell retention device. In a suspension culture system, the cell retention device can be a filter, but numerous other methods can be employed, such as sonic separation, inclined plane settling, external centrifuges, internal filters such as spinning or oscillating, external hydrocyclones, etc. The medium is provided, and the waste is removed, at a specified continuous flow rate when the perfusion system is activated.

Bone: Hard tissue formed by endochondral ossification and intramembranous ossification. Endochondral ossification involves the formation of the growth plate, a spatially organized structure within which chondrocytes mature through oriented proliferation, hypertrophy, and eventually either apoptosis or differentiation into osteoblasts. Intramembranous ossification involves the direct conversion of mesenchymal progenitors to osteoblasts without the intervening chondrocyte maturation or growth plate structure, and involves the gradual fusion of clusters of osteoblasts known as spicules. Mature bone is composed of three types of cells: osteoblasts, osteocytes, and osteoclasts. Osteoclasts, responsible for bone resorption, are derived from hematopoietic stem cells. Osteoblasts, responsible for bone synthesis, are derived from Sox9+ mesenchymal progenitors that can differentiate into chondrocytes or osteoblasts. Osteoblasts that become embedded within the bony matrix continue to differentiate into osteocytes. Osteocytes compose more than 90% of all mature bone cells and are involved in signaling to control calcium balance and bone remodeling in response to mechanical and hormonal cues via control of osteoblast and osteoclast differentiation. Osteoblastic cells lay down a calcified bony matrix composed primarily of type I collagen. Calcification requires the expression of alkaline phosphatase (Alpl) to provide the necessary phosphate for forming hydroxyapatite along with a host of matrix proteins that support the formation of calcified matrix including osteonectin, integrin binding sialoprotein (Ibsp), and osteopontin. Mammals are capable of complete and perfect regeneration of bone following fracture. The majority of fractures are healed by a combination of intramembranous and endochondral ossification. Following a fracture there is a brief inflammatory period after which periosteal and bone marrow mesenchymal precursors migrate to the site of fracture to initiate formation of a callus that is later remodeled to mature bone.

Bone-forming cells and mineral forming cells: Cells having osteogenic potential. Examples include, but are not limited to: bone marrow stromal cells, adipose-derived stem cells, osteoblasts, osteocytes, and dental pulp cells. “Osteogenesis” is the formation or production of bone. “Osteogenic” cells are cells (such as osteocytes or their precursors) capable of forming or producing bone. The precursor cells may be cells that have entered committed developmental pathways to be osteocytes. The osteogenic cells may or may not be present in association with already formed bone.

Cell Culture: The maintenance of cells in an artificial, in vitro environment that favors growth and survival. Suspension cell culture is a cell culture in which the majority or all of cells in a bioreactor, such as a culture vessel, are present in suspension (freely floating in liquid phase media), and the minority (or none) of the cells are attached to a surface.

Chondrocyte: Cells found in cartilage that act to produce and maintain the cartilaginous matrix. Chondrocytes produce all of the structural components of cartilage, including collagen, proteoglycans and glycosaminoglycans. Chondrocytes can be found as individuals or in clusters called isogenic groups, which represent recently divided cells. “Chondrogenic” cells that are capable of forming chondrocytes or cartilage, such as cells that have entered a developmental pathway that has committed them to be chondrocytes. “Cartilaginous” tissue refers to tissue that is partially, completely or substantially made of cartilage.

Electrical stimulus: The passing of various types of current or voltage selectively through one or more electrodes to a target location in a subject (for example, specific areas of the dorsolateral spinal cord).

Electrode: An electric conductor through which an electric current can pass. An electrode can also be a collector and/or emitter of an electric current. In some implementations, an electrode is a solid and comprises a conducting metal as the conductive layer. Non-limiting examples of conducting metals include noble metals and alloys, such as stainless steel and tungsten. An array of electrodes refers to a device with at least two electrodes formed in any pattern. A multi-channel electrode includes multiple conductive surfaces that can independently activated to stimulate or record electrical current.

Functional contact: Tissues or cells that are in with each other need not be in physical contact, but can be separated by an intermediate layer that mediates or allows biochemical communication between the tissues. For example, a layer of mesenchymal stem cells between a layer of chondrocytes and osteoblasts can physically separate, but still permit biochemical communication, between the chondrocyte and osteoblast layers.

Hypoxia: A condition of lower oxygen tension with respect to the oxygen tension of another tissue or fluid. For example, the oxygen tension of synovial fluid in humans is reported to normally be 6-9%. Atmospheric tension of oxygen is approximately 20%. Hence a fluid having an oxygen content lower than atmospheric is considered hypoxic with respect to atmosphere. The term “hypoxic” can also refer to relative oxygenation of different types of fluids in the body. A fluid, such as a fluid nutrient medium, can be made hypoxic with respect to another such medium, by adding an inert gas such as to the atmosphere in which a fluid is maintained.

Infrapatellar Fat Pad (IPFP): An anatomically intra-articular but extrasynovial adipose tissue. The IPFP contributes to the distribution of synovial fluid and force absorption in the joint, and damage by impingement results in a painful disease (Hoffa's Syndrome). The IPFP is highly vascularized and innervated with abundant adipocytes. The role of IPFP, long considered only as a cushion, was seldom considered in joint diseases. With the discovery of leptins, adipose endocrine functions and involvement in many physiologic and pathologic processes are now well recognized. Other major tissues also contribute to joint movement, including muscle, meniscus, intraarticular ligament, and their injuries are closely linked to onset of OA, most often resulting from abnormal mechanical loading that impacts cartilage and bone. Patients with knee OA typically present with reduced ability for full and voluntary muscle activation, but the underneath mechanism is still not clear.

Induced pluripotent stem cells (iPSCs): Cells generated by reprogramming 5 a somatic cell by expressing or inducing expression of a combination of factors (herein referred to as reprogramming factors). iPSCs can be generated using fetal, postnatal, newborn, juvenile, or adult somatic cells. In certain examples, factors that can be used to reprogram somatic cells to pluripotent stem cells include, for example, Oct4 (sometimes referred to as Oct 3/4), Sox2, c-Myc, 10 and Klf4, Nanog, and Lin28. In some examples, somatic cells are reprogrammed by expressing at least two reprogramming factors, at least three reprogramming factors, or four reprogramming factors to reprogram a somatic cell to a pluripotent stem cell.

Neuron: an electrically excitable cell that can produce an electrical signal (e.g., an action potential). Also called nerve cells. “Sensory neurons” are nerve cells within the peripheral nervous system responsible for converting stimuli from the environment of the neuron into internal electrical impulses and transmitting the impulse to the central nervous system. Also known as afferent neurons.

Normoxia: Normoxic atmosphere conditions are typically characterized by oxygen tensions between 10 and 21%. Normoxia as applied to different bodily fluids refers to the normal oxygen content (for example oxygen tension or saturation) of that bodily fluid (such as synovial fluid or whole blood or blood serum).

Nutrient fluid: A liquid, such as a medium, that supplies nutrients to living cells, such as a culture medium or fluid. Some such media are specialized to support the growth of a particular type of tissue, such as cartilage (cartilage media) or bone (bone media) or the cells contained in such tissue. A nutrient fluid can also be a fluid that normally supplies nutrients (such as oxygen) to a biological tissue. An example is synovial fluid that bathes the synovium in a mammalian joint.

Osteoarthritis: Cartilage damaged by trauma, disease or aging demonstrates very limited capabilities for self-regeneration and ultimately results in OA. Severe OA ultimately require total joint arthroplasty, a major surgery that completely ends the biological life of joint tissues. During the onset and progress of OA, structural, biophysical, biochemical and biomechanical changes are observed in joint tissues. Physical stresses such as mechanical overloading or traumatic injury likely play key roles, by acting either directly on cartilage and chondrocytes, or affecting other components first, with secondary damage to cartilage. Genetic factors also play a role in disease susceptibility. In general, OA often starts with pathologic activation of resident chondrocytes, followed by production of pro-inflammatory factors and other degradative enzymes. Osteophytes in the subchondral bone appear before measurable articular cartilage thickness changes as well as related joint space narrowing, suggesting earlier pathogenic events. The development of disease-modifying medications (DMMs) has targeted the subchondral bone, including antiresorptives, bone-forming, or dual function anti-osteoporotic agents.

Current pharmacological management protocols, including use of non-steroidal anti-inflammatory drugs (NSAIDs), specific inhibitors of cyclooxygenase-2, and intra-articular injection of cortiocosteroids or hyaluronan, are focused only on pain relief and joint function improvement. However, the underlying structural damage of the joint is not restored by these treatments, and long-term usage of NSAIDs has been shown to be associated with serious side-effects. While available surgical interventions exist, such as microfracture and osteochondral grafting, they are limited by formation of inferior fibrocartilage and donor tissue site morbidity, respectively.

Osteoblast: A mononucleate cell that is responsible for bone formation. Osteoblasts produce an osteoid matrix, which is composed mainly of collagen type I. Osteoblasts are also responsible for mineralization of the osteoid matrix. Bone is a dynamic tissue that is constantly being reshaped by osteoblasts, which build bone, and osteoclasts, which resorb bone. Osteoblasts arise from osteoprogenitor cells located, for example, in the periosteum and the bone marrow. Osteoprogenitors are immature progenitor cells that express the master regulatory transcription factor Cbfa1/Runx2. Once osteoprogenitors start to differentiate into osteoblasts, they begin to express a range of markers including osterix, collagen type 1, alkaline phosphatase, osteocalcin, osteopontin, and osteonectin.

Osteoclast: A type of bone cell that removes bone tissue by removing its mineralized matrix by a process of bone resorption. Osteoclasts are formed by the fusion of cells of the monocyte-macrophage cell line. Osteoclasts are characterized by high expression of tartrate resistant acid phosphatase and cathepsin K.

Osteocyte: Mature, non-dividing bone cells that are housed in their own lacunae (small cavities in the bone). Osteocytes are derived from osteoblasts and they represent the final stage of maturation of the bone cell lineage. While osteocytes are metabolically less active than osteoblasts, they serve as the principal mechanosensing cells in bone, and are responsible for regulating the activity of bone-building osteoblasts and bone-resorbing osteoclasts in response to mechanical loading. The narrow, cytoplasmic processes of osteocytes remain attached to each other and to osteoblasts through canaliculi (small channels in the bone).

Osteoconduction: The scaffold function provided by the transplanted matrix biomaterial which facilitates cell attachment and migration, and therefore the distribution of a bone healing response throughout the grafted volume. This property is likely dependent on extracellular matrix molecules, such as those found in bone matrix, including collagens, fibronectin, vitronectin, osteonectin, osteopontin, osteocalcin, proteoglycans and others. Growth factors in the matrix may also play a role.

Pain: An unpleasant sensory and emotional experience associated with actual or potential tissue damage, or described in terms of such damage. Pain experienced by mammals can be divided into two main categories: acute pain (or nociceptive) and chronic pain which can be subdivided into chronic inflammatory pain and chronic neuropathic pain. Acute pain is a response to stimulus that causes tissue injury and is a signal to move away from the stimulus to minimize tissue damage. Chronic pain, on the other hand, develops as a result of inflammation caused by tissue damage (inflammatory pain) or by damage to the nervous system such as demyelination (neuropathic pain). Chronic pain is generally characterized by stimulus-independent, persistent pain or by abnormal pain perception triggered by innocuous stimuli. Non-limiting examples of pain include postsurgical pain, pain associated with tissue damage, pain from inflammation, pain from infection (shingles), pain from neuropathic conditions, and pain from skeletal muscular conditions.

Perturbation: A disruption, such as in a culture. A perturbation can be chemical, mechanical or biological. A “perturbation” can be used to mimic a disease condition, such as osteoarthritis.

Pluripotent: The property of a cell to differentiate into all other cell types in an organism, with the exception of extraembryonic, or placental, cells. Pluripotent stem cells are capable of differentiating to cell types of all three germ layers (e.g., ectodermal, mesodermal, and endodermal cell types) even after prolonged culture.

Pluripotent stem cells: Stem cells that: (a) are capable of differentiating into teratomas when transplanted in immunodeficient (SCID) mice; (b) are capable of differentiating to cell types of all three germ layers (e.g., can differentiate to ectodermal, mesodermal, and endodermal cell types); and (c) express one or more markers of embryonic stem cells (e.g., express Oct 4, alkaline phosphatase, SSEA-3 surface antigen, SSEA-4 surface antigen, nanog, TRA-1-60, TRA-1-81, SOX2, REX1, etc.), but that cannot form an embryo along with its extraembryonic membranes (are 15 not totipotent). Exemplary pluripotent stem cells include embryonic stem cells derived from the inner cell mass (ICM) of blastocyst stage embryos, as well as embryonic stem cells derived from one or more blastomeres of a cleavage stage or morula stage embryo (optionally without destroying the remainder of the embryo). These embryonic stem cells can be generated from embryonic material produced by fertilization or by asexual means, including somatic cell nuclear transfer SCNT), parthenogenesis, and androgenesis. PSCs alone cannot develop into a fetal or adult animal when transplanted in utero because they lack the potential to contribute to all extraembryonic tissue (e.g., placenta in vivo or trophoblast in vitro). Pluripotent stem cells include iPSC generated by reprogramming a somatic cell by expressing or inducing expression of a combination of factors (herein referred to as reprogramming factors).

Polydimethylsiloxane (PDMS): a polymer compound with chemical formula CH₃[Si(CH₃)₂O]ₙSi(CH₃)₃.

Stem cell: A cell that under suitable conditions is capable of differentiating into a diverse range of specialized cell types, while under other suitable conditions is capable of self-renewing and remaining in an essentially undifferentiated pluripotent state. The term “stem cell” also encompasses a pluripotent cell, multipotent cell, precursor cell and progenitor cell. Exemplary human stem cells can be obtained from hematopoietic or mesenchymal stem cells obtained from bone marrow tissue, embryonic stem cells obtained from embryonic tissue, or embryonic germ cells obtained from genital tissue of a fetus. Exemplary pluripotent stem cells can also be produced from somatic cells by reprogramming them to a pluripotent state by the expression of certain transcription factors associated with pluripotency; these cells are called “induced pluripotent stem cells” or “iPSCs”.

Synovium: A specialized connective tissue that serves as the joint lining capsule. A healthy synovium consists of a thin intimal lining layer of fibroblast- and macrophage-like synoviocytes (FSs and MSs) and macrophages, and a sub-lining layer of loose connective tissue. MSs are able to remove wear-and-tear tissue debris, and FSs produce large amount of hyaluronan and other matrix proteins, which together maintain the health of the synovial fluid. Under certain conditions, such as infection and over exposure to tissue debris, the synovium can become irritated and thickened (synovitis), accompanied by increased macrophage recruitment and invasion of other inflammatory cells. Consequently, the normal function of the joint is compromised, such as pain and reduced mobility.

Tissue Culture Medium or Medium: A synthetic set of culture conditions with the nutrients necessary to support the growth (cell proliferation/expansion) and survival of a specific population of cells. Tissue culture media generally include a carbon source, a nitrogen source and a buffer to maintain pH. In one example, a medium contains a minimal essential media, such as DMEM, supplemented with various nutrients to enhance stem cell growth. Additionally, the minimal essential media may be supplemented with additives such as horse, calf or fetal bovine serum. A “chemically defined” cell culture medium is one in which each chemical species and its respective quantity is known prior to its use in culturing cells. A chemically defined cell culture medium is made without lysates or hydrolysates whose chemical species are not known and/or quantified. The terms “serum-free culture conditions” and “serum-free conditions” refer to cell culture conditions that exclude serum of any type. These terms can be used interchangeably.

Tissue Scaffold: A support that maintains mammalian cells in a three-dimensional matrix and allows for perfusion or bathing of nutrient fluids (for example, tissue culture medium) through the support to supply the cells. Non-limiting examples include synthetic scaffolds, such as polymer scaffolds, and non-synthetic scaffolds, for example pre-formed extracellular matrix or a de-cellularized organ scaffold. A scaffold can be in a particular shape or form so as to influence or delimit a three-dimensional shape or form assumed by a population of mammalian cells (such as proliferating mammalian cells). In some examples the scaffold is a thin three-dimensional substrate having opposite faces that can be separately bio-printed or seeded with cells. For example, the opposite surfaces may be parallel to one another and the outline of the scaffold as viewed from above may be any shape, such as circular, elliptical, oval, or polygonal (for example a rectangle, such as a square).

### II. Neu-MicroJoint Bioreactor

Provided herein is joint-on-a-chip tissue bioreactor (neu-microJoint), that integrates engineered osteochondral complex, synovium, adipose tissue, and nerve cells, enabling interrogation of the dynamic interplay between the peripheral nervous system and joint tissues. By recording activity in sensory neurons, joint integrity as well as therapeutic efficacy can be monitored in real time.

FIG. 1 depicts the cellular organization of the neu-microJoint bioreactor. Adipose, synovium, and cartilage/bone cells are contained within three difference chambers that are innervated with sensory neurons located in a separate chamber. Nutrient fluid (e.g., tissue culture media) flows between the chambers. The cell bodies of the sensory neurons are in a central chamber, and neurites of sensory neurons extend though microchannels to the adjacent tissue chambers containing the adipose, synovium, and cartilage/bone cells.

The bioreactor includes mammalian cells, such as human or veterinary cells. Other joint components, such as meniscus, ligaments and nerve may also be included in separate bioreactor chambers incorporated into the tissue bioreactor.

In some examples, the tissue bioreactor models known stratifications and physiologic conditions in human OA, inflamed arthritis and diabetic-induced complications of diabetes and other joint diseases (for example in the knee) to study and mimic the cause of onset, effect on target tissue elements and disease progression. In further examples, the tissue bioreactor models a whole joint, and is suitable for the initiation or acceleration of joint diseases with different pathophysiologic mechanisms to allow the investigation of disease onset and progression, the development of therapeutics that target different tissues and/or pathways, and the assessment of direct and indirect effects of candidate drugs.

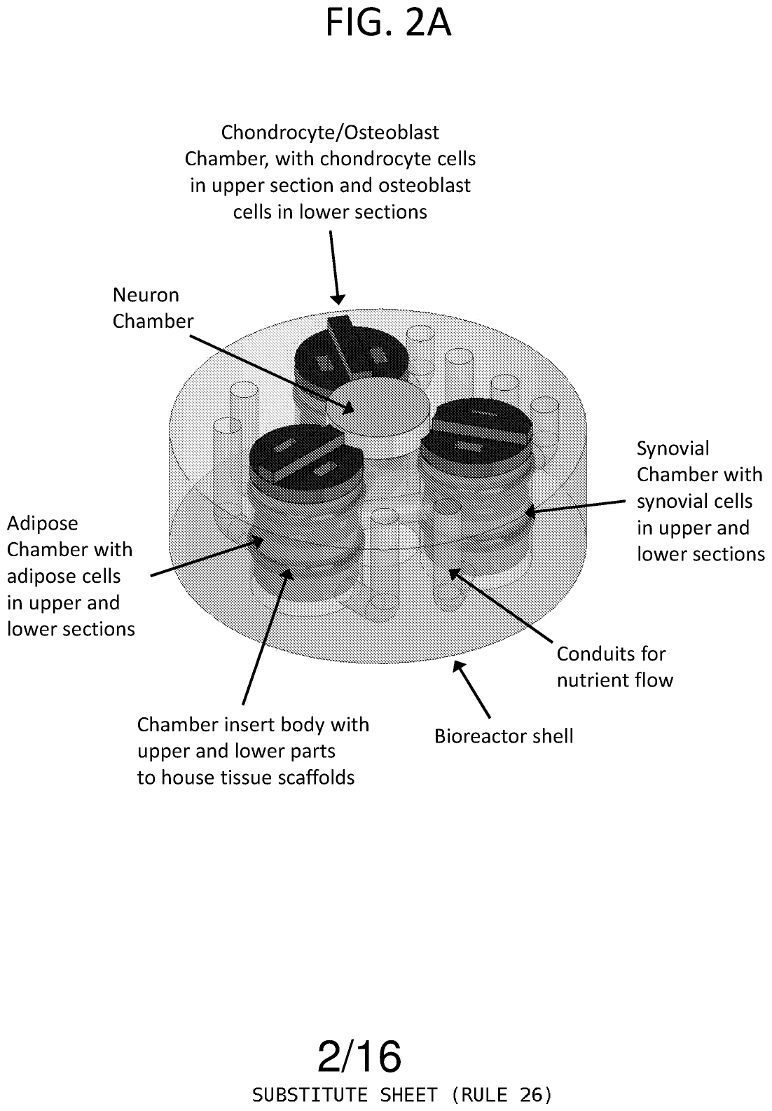

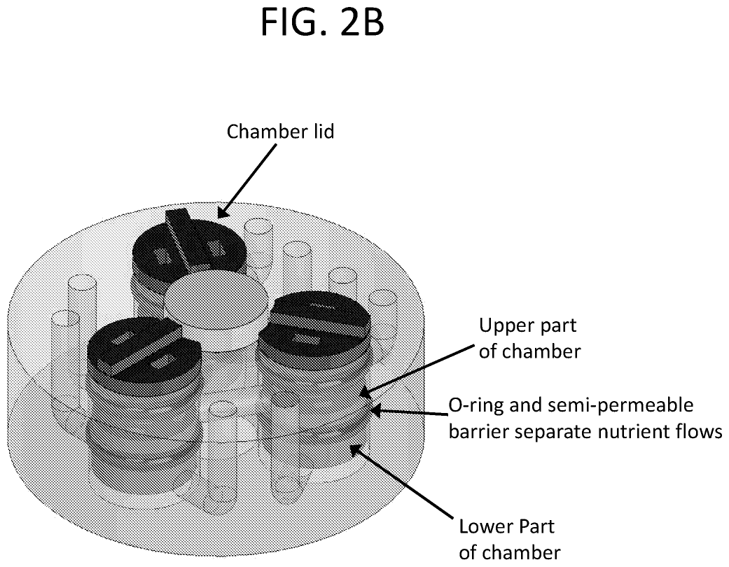

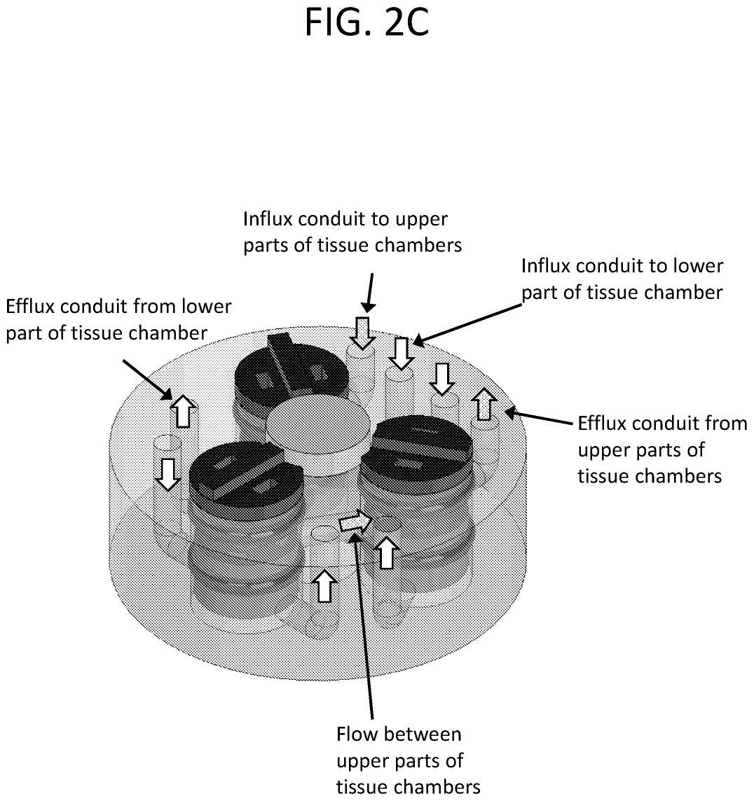

Referring to FIG. 2, an exemplary neu-microJoint bioreactor is provided.

FIG. 2A shows a three-dimensional rendering of an exemplary neu-microJoint bioreactor, illustrating the cell/tissue chambers and conduits for nutrient flow. The neu-microJoint bioreactor includes a shell forming four tissue chambers. The four chambers are a central chamber containing sensory neurons, and three outer chambers, containing adipose tissue, synovial tissue, and chondrocyte and osteoblast tissue. The outer tissue chambers are appropriately sized to receive chamber inserts that contain tissue construct(s) and separate the individual chambers into upper and lower parts. The upper and lower parts of each of the outer chambers are connected to fluid conduits in the shell that provide separate fluid flows to the lower parts of each outer chamber, and a single flow path linking the upper parts of the outer chambers. The shell can be made of any material suitable for incubating and growing cells, for example, glass, metal, or plastic.

Referring to FIG. 2B, the three outer tissue chambers each have an upper part and a lower part, which are separated by a barrier layer that prevent cell migration between the upper and lower parts of the chamber but allows biochemical communication between the tissue constructs in the two chambers, that is, the tissue constructs are in functional contact. The barrier layer can be, for example, a semi-permeable membrane having suitable pore sizes (e.g., about 20 μm pores) in the chamber insert, or a heterologous tissue scaffold, for example, composed of an additional MSC or iPSC layer. Additionally, an o-ring (present on the chamber body insert or located on the shell) seals the chamber body insert to the wall of the shell, which in combination with the semi-permeable barrier, separates the chamber into the upper and lower parts.

FIG. 2C illustrates the flow of fluid through the conduits of the neu-microJoint bioreactor. Hollow arrows indicate flow of nutrient fluid to the lower parts of individual tissue chambers. Each of the outer chambers has an influx and an efflux conduit supplying fluid to the lower part of the chamber. This allows separate nutrient fluids to be used of the lower parts of each of the outer chambers. Shaded arrows indicate the flow of nutrient fluid through the upper parts of the three tissue chambers in series. Fluid enters a first outer chamber via an influx conduit, then flows from the upper part of the first outer chamber via an interconnect conduit to the upper part of the second outer chamber, then through a separate interconnect conduit to the upper part of the third outer chamber, and finally exits the neu-microJoint bioreactor via an efflux conduit. The fluid can be recirculated back to the influx conduit supplying the first chamber. This allows the fluid flowing through the upper chambers to be in functional contact with the tissues in each of the chambers. Movement of the fluid through the neu-microJoint bioreactor can be accomplished with any suitable means, such as the use of pumps connected to appropriate tubing that is sealingly connected to the influx and efflux conduits.

This design allows for the provision of different fluids, compounds, and nutrients (e.g., a tissue culture medium or nutrient broth such as serum, or various other growth factors, steroids, growth hormones, etc.), or different concentrations of such materials, to the upper and lower parts of the chambers, and thus to different biological tissue layers disposed within the chambers. In some cases, the specific fluids and nutrients used can be tailored to the particular cell types grown in the bioreactor reactor chamber, as discussed below. For example, hypoxic fluids can be fed through the upper chamber while normoxic fluids are fed through the lower chamber.

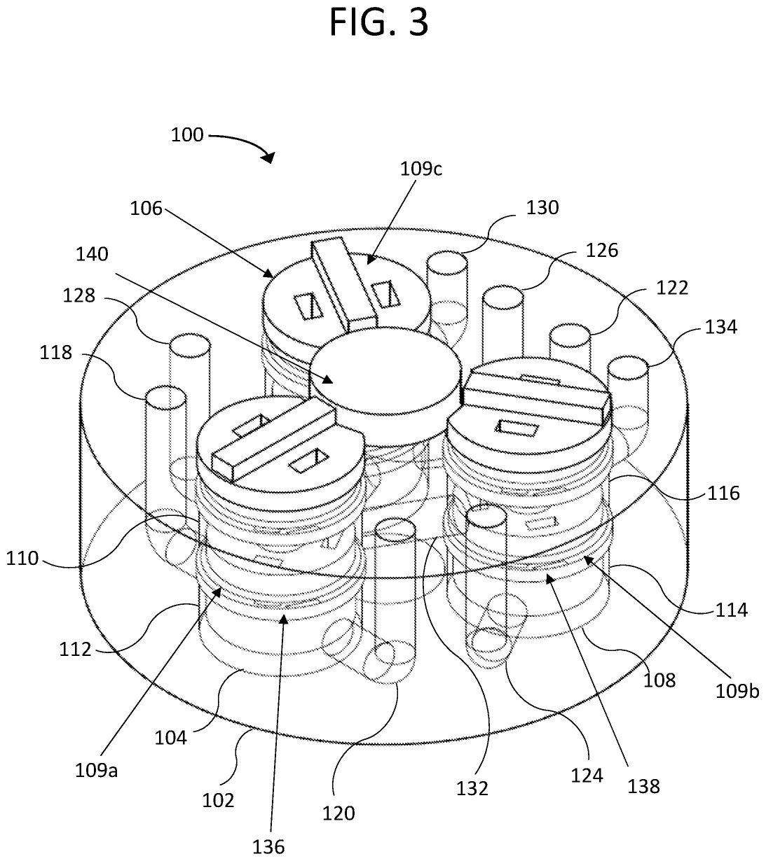

FIG. 3 provides additional detail for an example of the neu-microJoint bioreactor. The neu-microJoint bioreactor 100 has a shell 102 forming three outer chambers 104, 106, and 108, and one central chamber 110. The outer chambers 104, 106, and 108, each comprise a chamber body insert 109 containing at least one tissue construct, for a total of three chamber body inserts 109a, 109b, and 109c. The chamber body inserts 109a, 109b, 109c have substantially the same structure, but comprise different tissue constructs, as discussed herein. With the chamber body inserts present, the outer chambers 104, 106, and 108, each have an upper part and a lower part. Outer chamber 104 has upper part 110 and lower part 112. Outer chamber 108 has upper part 114 and lower part 116. The upper and lower parts of chamber 106 are not shown in the figure.

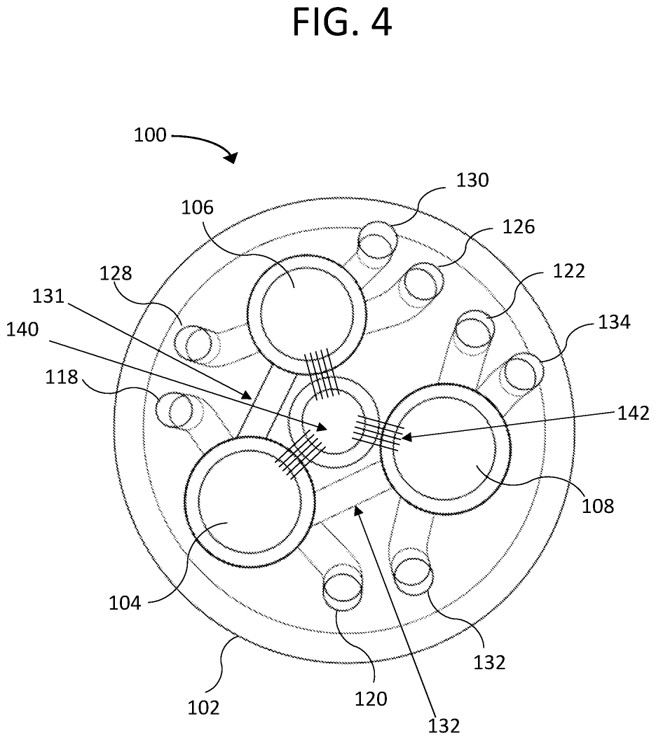

The upper and lower parts of the chambers are connected to different fluid conduits. Influx and efflux conduits 118 and 120 connect to the lower part 112 of chamber 104. Influx and efflux conduits 122 and 124 connect to the lower part 114 of chamber 108. Influx and efflux conduits 126 and 128 connect to the lower part of chamber 106. Regarding the upper parts of the chambers, fluid enters via influx conduit 130, flows to the upper part of tissue chamber 106 then to the upper part of tissue chamber 104 via an interconnect conduit 131 (see FIG. 4), then to the upper part of tissue chamber 108 via interconnect conduit 132, and then exits the neu-microJoint bioreactor via efflux conduit 134. Although the conduits are labeled “influx” and efflux” it will be appreciated that fluid flow can go in either direction.

When the neu-microJoint bioreactor is in use:
influx conduit 118 supplies a first nutrient fluid to the lower part of the outer chamber 104, and an efflux conduit 120 removes the first nutrient fluid from the lower part of the outer chamber 104;
influx conduit 122 supplies a second nutrient fluid to the lower part of the outer chamber 108, and efflux conduit 124 removes the second nutrient fluid from the lower part of the outer chamber 108;
influx conduit 126 supplies a third nutrient fluid to the lower part of the outer chamber 106, and efflux conduit 128 removes the third nutrient fluid from the lower part of the outer chamber 106; and
influx conduit 130 supplies a fourth nutrient fluid to the upper part of outer chamber 106, the nutrient fluid travels to the upper parts of outer chambers 104 and 108 via interconnect conduits 131 and 132, and efflux conduit 134 removes the fourth nutrient fluid from the upper parts of outer chamber 108. Thus, the fourth nutrient fluid passes through the upper part of all three outer chambers.

The upper and lower parts of the outer chambers are separated by a barrier located in the chamber body insert and the seal between the chamber body insert and the wall of the outer chamber. The barrier can be, for example, a semi-permeable barrier such as a tissue layer comprising mesenchymal stem cells or a semi-permeable synthetic membrane (or combination thereof). An o-ring (present on the chamber body insert or located on the shell) seals the chamber body insert to the wall of the shell to separate the chamber into the upper and lower parts. The o-ring can be made of any material suitable for use in a bioreactor that creates a fluid-tight seal between the wall of the chamber and the chamber tissue insert body. In FIG. 3, o-ring 136 seals chamber 104 into upper and lower parts. O-ring 138 seals chamber 108 into upper and lower parts. The o-ring for chamber 106 is not shown.

The semi-permeable barrier and the o-ring separate the upper and lower parts of the outer chambers to prevent bulk transfer of fluid between the upper and lower parts of the chambers. This allows for selective perfusion of the lower part of each chamber with nutrient fluids optimized for the particular tissue in the lower chamber. For example, the nutrient fluid used for the lower part of each chamber can be selected to optimize growth of the tissue construct in that lower part, without needing to consider any effect on other tissue constructs located in other chambers.

The neu-microJoint bioreactor contains central chamber 140 containing the sensory neurons. Central chamber 140 is positioned adjacent to and in between the three outer chambers.

FIG. 4 provides a plan view of the neu-microJoint bioreactor 100, showing additional detail. Three sets of microchannels 142 (see FIG. 4) run from the central chamber 140 to the three outer chambers 104, 106, and 108 of the neu-microJoint bioreactor 100. The microchannels shown in FIG. 4 are not to scale. The microchannels are configured to allow growth of neurites of the sensory neurons in the central chamber to the lower parts of the outer chambers, and to limit bulk flow of fluids between the central and outer chambers. The microchannels can be positioned at any suitable location within the neu-microJoint bioreactor that allows for neurite outgrowth from the central chamber to the three outer chambers. For example, the microchannels are located at the floor of the central chamber, at points closest to the adjacent outer chambers, and run horizontally from the central chamber through the shell to the floor of the three outer chambers.

Any suitable number and size of microchannels can be included in a set of microchannels interconnecting the central chamber to an outer chamber. In some examples, there are from 5 to 100 microchannels interconnecting the central chamber with each of the outer chambers (that is, three sets of 5 to 100 microchannels). The length of the microchannels is determined by the distance between the central chamber and the relevant outer chamber. In some example the microchannels interconnecting the central chamber with the outer chambers are from about 50 μm-about 2000 μm in length. As used herein, “about,” in a quantitative context, means plus or minus 5% from a reference value. The microchannels can have any suitable cross-sectional shape, such as round, oval, square, or rectangular. Typically, the microchannels have a cross-sectional area of about 20 μm² to about 100 μm².

The microchannels travel through the neu-microJoint shell to connect the central chamber with the outer chambers. The microchannels can be formed using any suitable method. For example, if the shell is made using 3D printing, then the microchannels can be formed during the printing process.

The neu-microJoint shell can be constructed using any suitable process. In some examples, the shell is formed from a set of components bonded together, and the microchannels are formed at the joint between components. For example, the shell can be formed from an upper layer and a bottom layer, where the upper layer provides the walls of the central and outer chambers and the fluid conduits, and the bottom layer provides the bottom of the shell and the floor of the central and outer chambers. In this example the microchannels are located at the interface of the upper and bottom layers, adjacent to the floors of the central and outer chambers. The volume of the microchannels is formed in one of the layers, and enclosed when the layers are bonded together. In some examples, the microchannels are formed by coating and masking of a first shell layer during the production process; when the mask is removed, the resulting negative space (or grooves) forms the inner volume of the microchannel, and the final wall of the channel is provided when the second layer of the shell is bonded to the first.

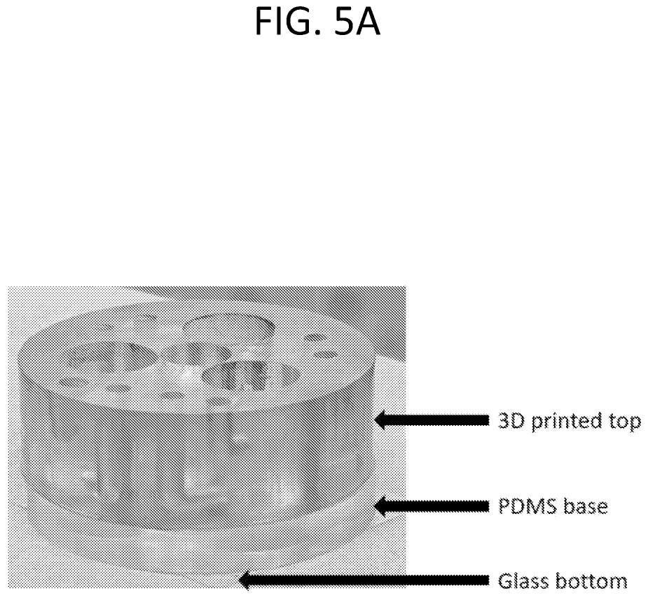

In some examples, the shell is formed from three components, an upper layer, a base layer, and a bottom layer. See, for example, FIG. 5A, which shows an exemplary shell for use with the neu-microJoint bioreactor provided herein. FIG. 5B shows schematic diagrams illustrating the dimensions of an example upper layer and base layer (sizes are in mm). The upper layer is 3D printed from biocompatible resin, which is then bonded to a base layer made of biocompatible material (such as PDMS). The 3D-printed upper layer contains the influx and efflux conduits, and hollow cylinders for the central and outer chambers. The base layer contains matching hollow cylinders for the central and outer chambers, and is bonded to the upper layer such that the central and outer chambers extend through the upper layer and the base layer. The bottom of the shell, and the floor of the central and outer chambers is provided by a bottom layer that is bonded to the base layer. Prior to attachment of the bottom layer, the lower surface of the base layer is treated (for example with a masking or etching process) to form partial walls (or grooves) of the microchannels between the central neuron chamber and the outer tissue chambers. When the bottom layer is joined to the base layer, the walls of the microchannels are completed.

In some examples, a flat glass surface is used for the bottom layer, such as a glass coverslip to facilitate live cell imaging.

In some examples, the bottom layer of the shell forming the bottom of the shell and the floor of the inner and outer chambers is made of glass and is suitable for microscopic imaging of cells in the chambers, such as the sensory neurons in the central chamber or neurites traveling through the microchannels. For example, calcium imaging of the sensory neurons or neurites.

In some examples, the central and/or outer chambers contain electrodes (such as one or more microelectrode arrays) suitable for stimulating or recording neural signals from the sensory neurons in the chamber. The electrodes (such as one or more microelectrode arrays) are typically located on the floor of the central chamber. In some examples, a stimulating electrode is embedded under the microchannels to enable the generation of orthodromically conducted action potentials in neurons extending neurites into the other tissue compartments. During use, the electrodes are coupled to recording and/or stimulating circuitry. Coupling of the circuitry to the electrode can be by way of one or more leads, although any operable coupling capable of transmitting the measured neural signal from the electrode sites to the circuitry, or a stimulation signal from the circuitry to the electrodes, can be used.

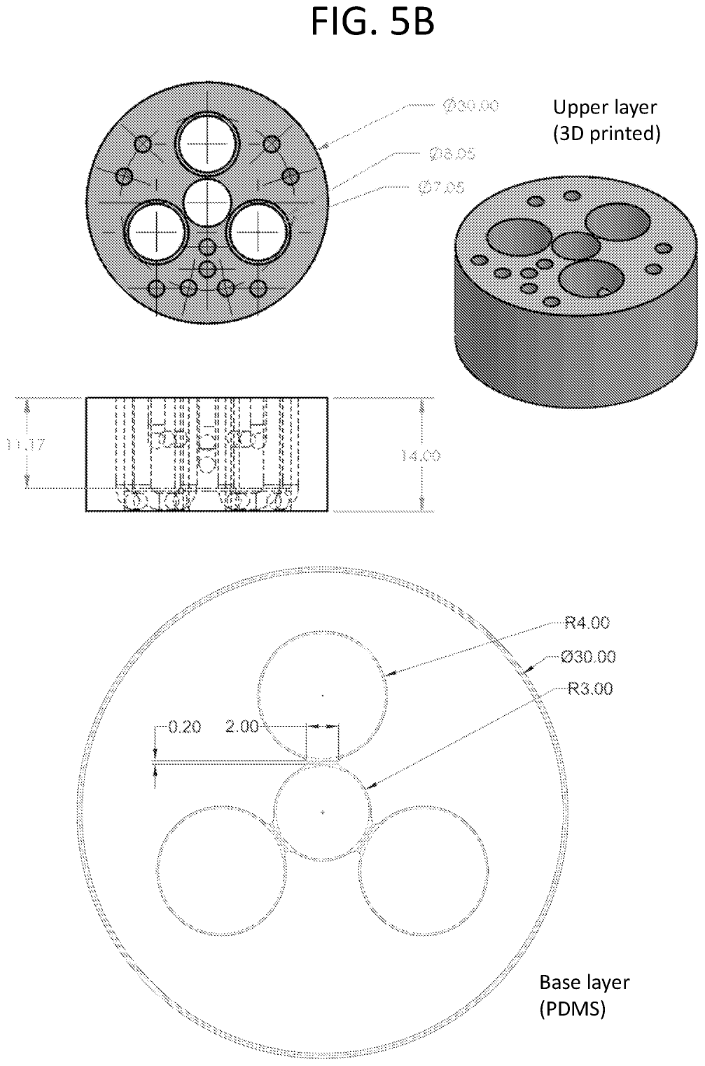

Referring to FIG. 6, optionally, the neu-microJoint bioreactor includes additional influx and efflux conduits to provide fluids to the central chamber containing the sensory neurons. FIG. 6 shows the shell 202 of a neu-microJoint bioreactor 200 that contains all the features of the shell 102 of neu-microJoint bioreactor 100 and additionally comprises influx conduit 206 leading to the central chamber 204 and an efflux conduit 208 leading from the central chamber 204. Influx conduit 206 supplies a nutrient fluid to the central chamber 204, and an efflux conduit 208 removes the nutrient fluid from the central chamber 204.

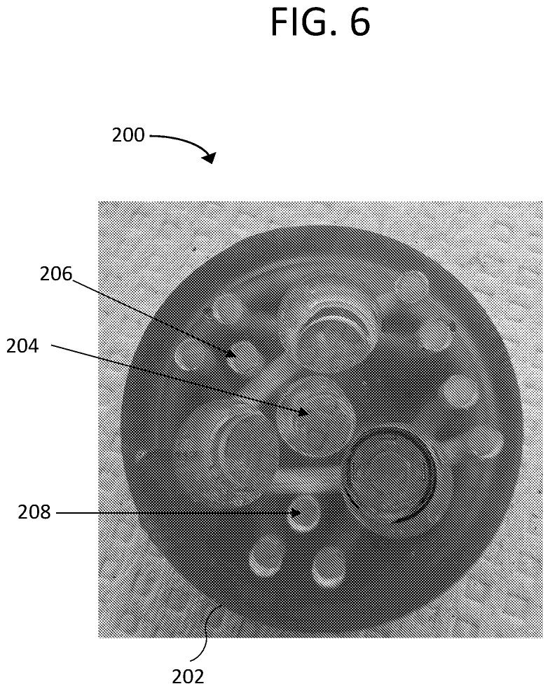

FIG. 7 shows an exemplary inner chamber body 302, and an exemplary upper ring 204, of chamber body insert for use in the neu-microJoint bioreactor as described herein. The inner body 302 includes a lower porous screen 306 and an upper porous screen 308, both of which include a plurality of pores, or small openings, 310. The inner body 302 also includes a protruding ring 312 which protrudes radially outwardly from the rest of the inner body 302, and which has an outside diameter approximating the inner diameter of the outer chambers of the neu-microJoint as described herein. Thus, when the inner body 302 is situated within an outer chamber of the neu-microJoint, several distinct chambers can be formed, as described above with regard to bioreactor reactor chamber 100.

FIG. 7 also shows that upper ring 304 has a groove 314 extending around the circumference of the inner surface of one end of the upper ring 304. The upper ring also has a main inner surface 316 having a generally cylindrical shape and an inner diameter approximating an inner diameter of the inner cylindrical space 318 in the inner body 302.

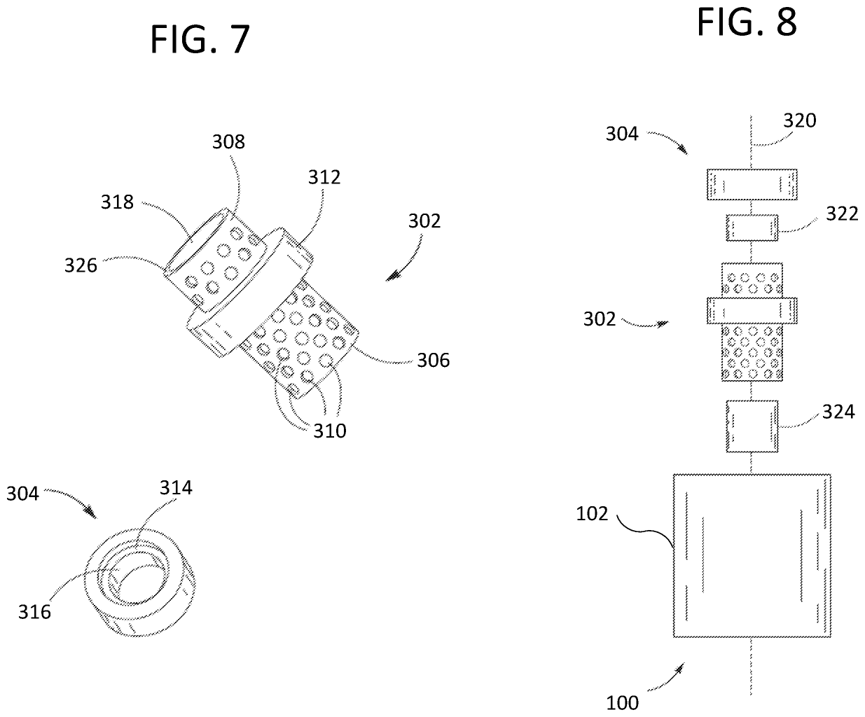

FIG. 8 shows a depiction of the shell 102 of neu-microJoint bioreactor 100, inner body 302, upper ring 304, upper tissue construct 322, lower tissue construct 324, aligned along axis 320 in an exploded view. These elements can be combined, to form one of the chamber body inserts containing tissue constructs and present in the neu-microJoint bioreactor 100, as described herein. When these components are assembled to form a bioreactor reactor chamber in this manner, the upper tissue construct 322 and lower tissue construct 324 are situated within the inner space 318 within the inner body 302. Further, a top end portion 326 of the inner body 302 can be situated within the groove 324 of the upper ring 304 to facilitate sealing of the system.

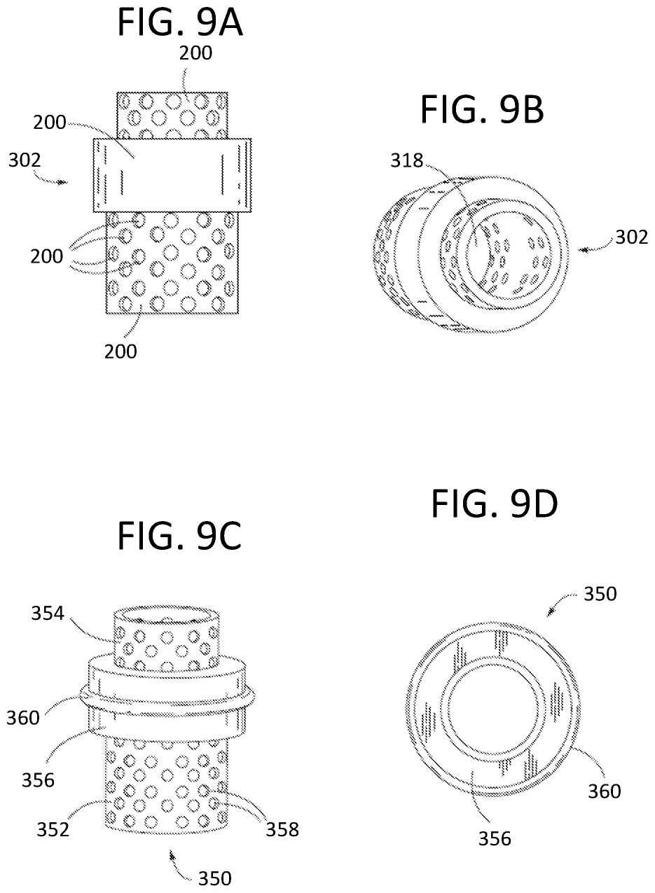

FIGS. 9A-9B show alternate views of the inner body 302 shown in FIGS. 7-8. FIG. 9B shows that the inner body 302 has a cylindrical inner open space 318 which spans through the entire body 302 to accommodate the positioning of cellular material therein. FIGS. 9C-9D illustrate an inner body 350 comprising a lower porous screen 352, an upper porous screen 354, and a protruding ring 356. The lower and upper porous screens have a plurality of pores 358. The inner body 350 also includes a sealing o-ring 360 disposed around the outside of the central protruding ring 356. The o-ring 360 helps seal the inner body 350 against the inner walls of the outer chambers of the neu-microJoint bioreactor to more effectively maintain distinct upper and lower parts of the chambers.

The inner body 302 and 350 can be fabricated, for example, photolithographically using a biocompatible plastic-polymer. In some examples, the shell, body and/or ring of an inner body, or other parts of a bioreactor reactor chamber, can be fabricated with commercially available E-SHELL 300™ polymer resin using photo-stereolithography (PSL).

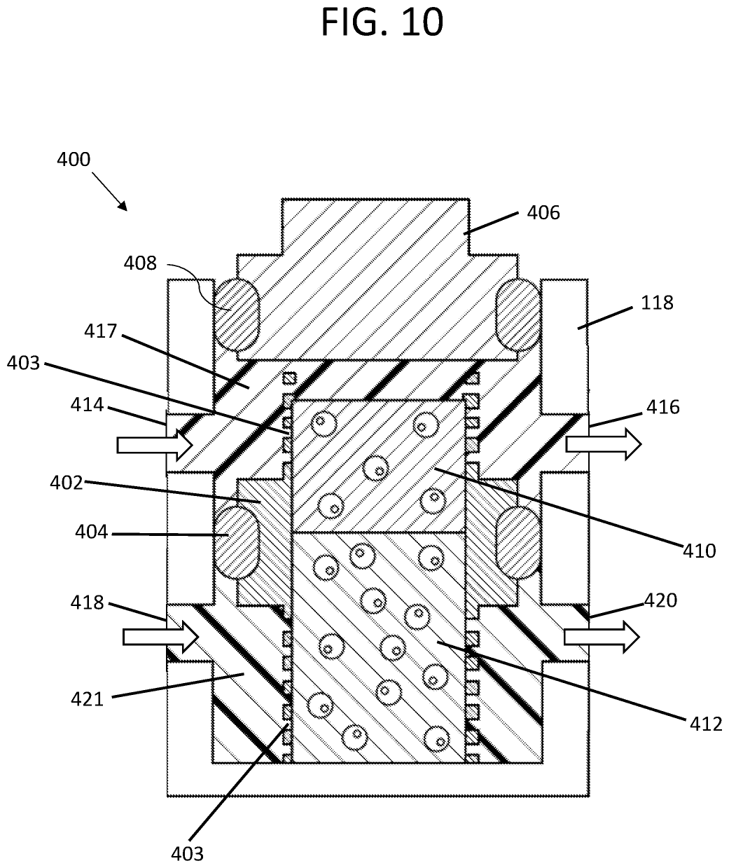

FIG. 10 shows a vertical plane cross-sectional view of a chamber 400 of an exemplary neu-microJoint bioreactor as described herein. An example of a chamber body insert with lid is present in the chamber, as are upper and lower tissue constructs, a barrier layer between the tissue constructs, and nutrient fluids. The chamber is configured to receive a chamber body insert and a sealing lid (the lid can be replaced with and/or incorporated into a mechanical actuator or piston that applies a mechanical loading pattern downward on the tissue/fluid in the bioreactor as a perturbation).

The chamber body insert 402 is sealingly engaged with the inner surfaces of the chamber 400 via an o-ring 404 to form separate upper and lower fluid flow chambers. The lid 406 is also sealingly engaged with the inner surfaces of the chamber 400 via another o-ring 408 to prevent fluid escaping from the chamber. The insert 402 can contain at least two tissue constructs, such an upper tissue construct 410 and a lower tissue construct 412 as shown. The upper tissue construct 410 can comprise chondrocytes within a scaffold (such as a gel matrix) and/or the lower tissue construct 412 can comprise a osteocytes within a scaffold (such as a gel matrix), for example. As shown, there is no physical separation between the upper tissue construction 410 and the lower tissue construct 412. However, there can be a semi-permeable membrane at this interface of the upper and lower tissue constructs. There can also be a third type of cells in a scaffold (such as a layer of mesenchymal cells in a scaffold) between the upper and lower tissue constructs.

The chamber has two opposing upper inlet/outlets 414 and 416, which allow a first fluid 417 to flow from influx and efflux conduits through the upper part of the chamber to interact with the upper tissue construct 410, and two opposing lower inlets/outlets 418 and 420, which allow a second fluid 421 to flow from influx and efflux conduits through the lower part of the chamber to interact with the lower tissue construct 412. In an example, the upper tissue construct 410 comprises chondrocytes and the first fluid comprises a chondrogenic medium, and the lower tissue construct 412 comprises osteocytes and the second fluid comprises an osteogenic medium, for example.

As illustrated in FIG. 10, the first fluid 417 can enter at 414 and then pass laterally through a perforations 403 in the chamber body insert 402 to enter the upper tissue construct 410 laterally. The first fluid 417 can then exit the upper tissue construct 410 laterally through the perforations in the chamber body insert 402 before exiting the bioreactor at 416. The perforations can extend circumferentially around the insert 402 such that the first fluid 417 can flow around the upper tissue construct 410 and can interact laterally with the upper tissue construct 410 from all lateral sides. Some of the first fluid 417 can also flow over the top of the upper tissue construct 410 and perfuse into and out of the upper tissue construct 410 from its upper surface. Similarly, the second fluid 421 can enter at 418 and then pass laterally through perforations 403 in the lower portion of chamber body insert 402 to enter the lower tissue construct 412 laterally. The second fluid can then exit the lower tissue construct 412 laterally through the perforations in the chamber body insert 402 before exiting the chamber at 420. The perforations can extend circumferentially around the lower portion of the chamber body insert 402 such that the second fluid can flow around the lower tissue construct 412 and can interact laterally with the lower tissue construct 412 from all lateral sides.

This design allows for the provision of different fluids, compounds, and nutrients (e.g., a tissue culture medium or nutrient broth such as serum, or various other growth factors, steroids, growth hormones, etc.), or different concentrations of such materials, to the upper and lower parts of the chamber, and thus to different biological tissue constructs disposed within the chamber. In some cases, the specific fluids and nutrients used can be tailored to the particular cell types grown in the bioreactor reactor chamber, as discussed herein. For example, hypoxic fluids can be fed through the upper part of the chamber while normoxic fluids are fed through the lower part of the lower chamber.

In some examples, systems capable of mechanically stressing the cellular material grown in a bioreactor are used to apply a perturbation to the cells in the bioreactor. Natural bone and cartilage growth is known to be affected by mechanical stresses encountered by those tissues as they grow, thus systems allowing the introduction of such stresses can facilitate tissue growth which more accurately resembles native tissue growth. In an example, the cap or lid of any one of the bioreactor reactor chambers can further include piston for example, as described in U.S. Pat. No. 11,339,362, incorporated by reference herein. The piston can be used to impart a compressive force on materials situated within the bioreactor reactor chamber.

The neu-microJoint bioreactor can be any suitable size or shape, such as shown in FIGS. 2-6. In some examples, the shell of the neu-microJoint bioreactor has a diameter of from 15 to 100 mm and a height 10 to 50 mm. In some examples, the central chamber has a diameter of from 5 to 20 mm and the outer chambers have diameters of from 5 to 20 mm. Any size and shape may be used that is suitable for placement of the central and outer chambers in sufficient proximity for neuritis to grow though the microchannels connecting the chambers, and for placement of the fluid conduits as described herein.

For use of the bioreactor, the shell is combined with the chamber inserts containing appropriate tissue constructs, the sensory neurons are incubated in the central chamber, and the fluid conduits are tapped and connected to appropriate tubing and pumps to flow nutrient fluids through the chambers.

### Cells and Tissues Scaffolds

The disclosed neu-microJoint bioreactor includes separate compartments containing chondrocytes, osteoblasts, synovial cells, adipose cells, and sensory neurons, which are integrated via a system of fluid conduits and microchannels to model the dynamic interplay between the peripheral nervous system and joint tissues and serve as a platform to interrogate joint pain and treatment thereof.

Any suitable cartilage, bone, synovium, adipose, and neuronal tissue or cells can be incorporated into the devices, systems, and techniques described herein, for example the cells and tissues disclosed in PCT Publication No. WO 2017/062629 and PCT Publication No. WO 2015/027186. These are mammalian, and can be human or veterinary. Physiologically relevant cartilage, bone, synovium and adipose tissues and/or cells can be obtained from primary tissue, cell lines, and/or generated from MSCs or iPSCs from the same individual or from different individuals. The major joint elements, such as bone, cartilage, synovium and adipose tissue, are typically engineered from human cells.

In some examples the cells of the disclosure are cultured within a scaffold. Typically, the tissue scaffolds are individually fabricated as modules that are introduced into a chamber body insert as provided herein and then inserted into the bioreactor shell. The fluid inlets and outlets in the different chambers are used to introduce culture media, as well as stimuli and therapeutics. In several examples, a culture has greater than 75%, 80%, 85%, 90%, 95%, 98%, or 99% of the cells within a scaffold. Any suitable scaffold can be used to culture cells, such as the scaffolds disclosed in PCT Publication No. WO 2017/062629 and PCT Publication No. WO 2015/027186.

In some examples the cells of the disclosure are cultured in a suspension cell culture. In several examples, a suspension culture has greater than 75%, 80%, 85%, 90%, 95%, 98%, or 99% of the cells in suspension, and thus not attached to a surface on or in the bioreactor.

In one example the cells grown in a chamber of the bioreactor are chondrocytes. In some examples the cells grown in the bioreactor are chondrogenic cells. In further examples the cells grown in the bioreactor, such as chondrocytes, form cartilage, or innervated cartilage.

In one example the cells grown in the bioreactor are adipose cells. In further examples the cells grown in the bioreactor are IPFP cells. In some examples the cells grown in the bioreactor, such as adipose cells, form fat pad tissue, IPFP tissue, innervated fat pad tissue, or innervated IPFP tissue.

In one example the cells grown in the bioreactor are osteoblasts. In some examples the cells grown in the bioreactor are osteoclasts. In further examples the cells grown in the bioreactor are osteocytes. In some examples the cells grown in the bioreactor, such as osteoblasts, osteoclasts, and/or osteocytes form bone tissue, or innervated bone tissue.

In one example the cells of the bioreactor are synovial cells. In some examples the cells of the bioreactor, such as synovial cells, form synovium or innervated synovium. In some examples the cells of the bioreactor, such as synovial cells, form joint lining capsule tissue or innervated joint lining capsule tissue.

The cells or tissues grown in the bioreactor can be derived from iPSCs or MSCs. Any suitable method of differentiating iPSCs or MSCs into the cells and tissues described herein can be used, such as the methods described in PCT Publication No. WO 2017/062629 and PCT Publication No. WO 2015/027186. In some examples, iPSCs are generated from human bone marrow stem cells (M-iPSCs), and MSC-like cells (iMPCs) are derived from the iPSCs thus obtained. The generated iMPCs can have chondrogenic, osteogenic, and adipogenic capabilities, and are used to produce human osteoblasts, chondrocytes and adipocytes, macrophages, and fibroblasts.

The components of a fabricated bioreactor platform can then be combined with these and/or other microtissue cellular components to assemble a bioreactor similar to bioreactor 100. Performance of the tissue compartments in the bioreactor can then be verified using, e.g., leakage tests, micro computed tomography (“mCT”), magnetic resonance imaging (“MRI”), MTS, Live/Dead, imaging, and/or histology/IHC techniques.

In some examples, the scaffold used for culturing cells is a hydrogel. Optionally, the hydrogel is a photocrosslinked gelatin hydrogel. In some examples, the hydrogel is a methacrylated gelatin hydrogel, such as a methacrylated hyaluronan hydrogel. The hydrogel can be a mixture of methacrylated gelatin and methacrylated hyaluronan hydrogel. The hydrogel can be a gelatin hydrogel, such as a methacrylated gelatin, and/or methacrylated hyaluronan hydrogel that was photocrosslinked with visible light.

In one specific non-limiting example, MSCs (4-20×10⁶/ml) are seeded in gelatin/hydroxyapatite hydrogels by photocrosslinking, and cultured in BMP-2 included osteogenic media. Cartilage is engineered by seeding MSCs (4-60×10⁶/ml) in gelatin/hyaluronic acid hydrogel by photocrosslinking, and treated with transforming growth factor-β 3 (TGF-β3) included chondrogenic medium. Osteochondral interfaces is formed by placing layers of MSC-laden (4-20×10⁶/ml) gelatin hydrogels between the chondral and osseous-constructs.

Where appropriate, the tissues used in the devices, systems, and methods described herein can be formulated with the use of scaffold crosslinking technologies, such as projection stereolithography (PSL) to incorporate internal 3D spatial features which permit optimal tissue formation and medium perfusion. For example, 500-micron-diameter channels can be fabricated within the bone construct to aid in nutrient dispersion throughout the construct. Bone can be formed by seeding and culturing mesenchymal stem cells (MSCs) in photocrosslinked collagen/hydroxyapatite. Collagen and hydroxyapatite, or Ca₁₀(PO₄)₆(OH)₂, are primary components of bone, and both are frequently used in tissue engineered bone constructs. Cartilage can be engineered by seeding MSCs in a photo-activated/crosslinked polymeric gel, such as a collagen/chitosan gel, and treated with TGF-β3. Chitosan can be advantageous, as it shares some structural characteristics with glycosaminoglycans, a critical component of cartilage responsible for many of its specific mechanical properties. With its many primary amine groups, chitosan can also aid in collagen crosslinking.

Osteochondral interfaces can be formed from a variety of cellular and other materials arranged in various combinations with one another. An exemplary osteochondral interface can be formed by placing a layer of MSC-laden collagen type I hydrogel between the chondral and osseous layers. The synovial lining can be generated with MSCs seeded in crosslinked polyethylene glycol alone and cultured in non-inductive medium. These conditions have been shown in experiments to be capable of maintaining a fibroblastic phenotype in MSCs. As previously mentioned, the endothelial component can comprise endothelial cells embedded in collagen to surround the osteochondral elements. Collagen gels can be selected based on their susceptibility to modification and contraction by endothelial cells and osteoblasts, which can result in a tight fit around the osteoblast construct.

As there are limited differentiated cell sources available for cartilage and bone tissue engineering, adult multipotent mesenchymal stem cells (MSCs), with their well-characterized ability to differentiate into chondrocyte- and osteoblast-like cells, represent an advantageous candidate cell source for engineering these tissues. Human MSCs derived from bone marrow or from adipose (lipoaspirate) can be used as the progenitor cell population to engineer the bone, cartilage, and synovium components of the microtissue. However, the microtissue system described herein is compatible with constructs derived from any type of progenitor or primary cell. Indeed, induced pluripotent stem cells, with their ability to be propagated to meet the high cell requirements of tissue engineering, represent an attractive, high-quality cell source and provide one exemplary alternative source.

Chondrocytes are a major cell type in cartilage. Other cells such as cartilage progenitor cells are also present, but their number is limited. Several tissue specific cell types reside in bone, including osteoblast, osteocyte, lining cells and osteoclast; both osteocytes and lining cells are related to osteoblasts.

The synovium contains fibroblast-like synoviocytes (FSs; 98%) and macrophages (2%), with the former considered as the major cell types responsible for OA pathogenic mechanisms.

For adipose tissues, cells can be divided into adipocytes and those in stromal capsular fraction (SVF), a heterogeneous mixture of adipose stromal cells (ASCs; 15-30%), endothelial cells, pericytes, and immune cells.

Engineered tissue constructs which properly incorporate plural tissue layers into an interactive microtissue unit can help in accurately studying biological tissues and their interactions, and can help in elucidating the pathogenesis of various diseases and assessing the efficacy of potential therapeutics against those diseases. Some of the devices, systems, and methods described herein facilitate the growth of physiologically accurate microsystems having distinct biological tissue layers, such as those found within an organ (e.g., the liver) or other physiological system (e.g., the skeletal system). Portions of the current disclosure refer to the osteochondral complex and OA, which are of particular interest herein, although the devices, systems, and methods disclosed should be understood to be applicable to multi-tissue cultures generally.

In some examples, chondrocytes, osteoblast and adipocytes are generated from human MSCs, and 3D osteochondral and adipose tissues are produced. MSC differentiation can then be verified using, e.g., histological and reverse transcription polymerase chain reaction (“RT-PCR”) techniques. In some examples, undifferentiated MSCs are encapsulated in a collagen type 1 gel to form a mesenchymal construct, or in PEG to form a synovium. In other examples, pre-differentiated osteoblasts are encapsulated in hydroxyapatite-containing collagen type 1 gel to form an osteoblast construct. In yet other examples, pre-differentiated chondrocytes are encapsulated in a collagen type 1/chitosan gel to form a chondrocyte construct. Endothelial cells can also be isolated and encapsulated in a collagen type 1 gel to form an endothelium.

Physiologically relevant cartilage, bone, synovium and adipose tissues can be generated from MSCs or iPSCs from the same individual or from different individuals. Chondrocytes are the major cell type in cartilage. Other cells such as cartilage progenitor cells are also present, but their number is limited. Several tissue specific cell types reside in bone, including osteoblast, osteocyte, lining cells and osteoclast; both osteocytes and lining cells are related to osteoblasts. The synovium contains fibroblast-like synoviocytes (FSs; 98%) and macrophages (2%), with the former considered as the major cell types responsible for OA pathogenic mechanisms. For adipose tissues, cells can be divided into adipocytes and those in stromal capsular fraction (SVF), a heterogeneous mixture of adipose stromal cells (ASCs; 15-30%), endothelial cells, pericytes, and immune cells. In addition to the tissue specific cells. nerve and vascular system are also found in joints, as in most tissues/organs. OA is associated with altered innervation patterns, generally considered as a consequence of other tissue pathological changes. Therefore, in some examples, the disclosed neu-microJoint bioreactor may also include functional nerve tissue. The microfluidic circulation included in the neu-microJoint bioreactor is equivalent to a functional vascular system. Generation of macrophages from human iPSCs had also been reported.

In some examples, chondrocytes, osteoblast and adipocytes are generated from human MSCs, and 3D osteochondral and adipose tissues are produced. MSC differentiation can then be verified using, e.g., histological and reverse transcription polymerase chain reaction (“RT-PCR”) techniques. In some examples, undifferentiated MSCs are encapsulated in a collagen type 1 gel to form a mesenchymal construct, or in PEG to form a synovium. In other examples, pre-differentiated osteoblasts are encapsulated in hydroxyapatite-containing collagen type 1 gel to form an osteoblast construct. In yet other examples, pre-differentiated chondrocytes are encapsulated in a collagen type 1/chitosan gel to form a chondrocyte construct. Endothelial cells can also be isolated and encapsulated in a collagen type 1 gel to form an endothelium.

The various microtissue cellular components thus formed (e.g., mesenchymal construct, synovium, osteoblast construct, chondrocyte construct, and endothelium) can then be verified for viability and tissue type, using, e.g., 3-(4,5-dimethylthiazol-2-yl)-5-(3-carboxymethoxyphenyl)-2-(4-sulfophenyl)-2H-tetrazolium (“MTS”), Live/Dead staining, and/or histology/immunohistochemistry (“IHC”) techniques.

Several biomaterials may be used to produce the tissue constructs included in the neu-microJoint bioreactor. In one example, given the prevalence of collagen in joint tissue matrix, the tissue constructs are engineered by encapsulating iPSC-derived joint cells or MSC-derived joint cells within a photo-crosslinkable methacrylated gelatin (mGL) in a top chamber. The mGL displays excellent biocompatibility and support for cell growth, and cartilage, bone and adipose tissues are successfully engineered from human iMPCs seeded within mGL, with differentiation occurring within three weeks. The top chamber containing the mGL encapsulating the iPSC-derived joint cells or MSC-derived joint cells is then placed on top of a bottom chamber containing a polycaprolactone scaffold that constitutes the osseous component to create a 3D biphasic osteochondral construct.

### Tissue Culture Media

Any suitable nutrient fluid can be circulated through the devices and systems described herein, for example the nutrient fluids disclosed in PCT Publication No. WO 2017/062629 and PCT Publication No. WO 2015/027186.

In screening candidate drugs that may modify, stop or reverse progression of joint pain (such as induced by OA), and that act either locally (synovial fluid) or systemically, a key consideration is that joint diseases not only involve all elements in one joint, but also often affects several joints at the same time. Therefore, both local and systemic factors should be considered. Accordingly, to accommodate both local and systemic environment, the disclosed neu-microJoint bioreactor allows for optimized culture conditions for different tissues located in different parts of the device.

A unique feature of the disclosed neu-microJoint bioreactor is that the lower parts of the tissue chambers are isolated from the rest of the device, and the circulating media can be different in each of the lower parts. Typically, tissue culture media circulated through the lower parts of the chambers is optimized for the particular type of cells or tissue in the lower part of the chamber.

The tissue media circulated through the upper parts of the tissue chambers in the bioreactor can proceed in any desired order. In one example, the nutrient fluid contacts synovium, cartilage, and adipose in that order. In one example, the nutrient fluid contacts cartilage, adipose, and synovium in that order. In one example, the nutrient fluid contacts adipose, synovium, and cartilage in that order. In one example, the nutrient fluid contacts cartilage, synovium, and adipose in that order. In one example, the nutrient fluid contacts synovium, adipose, and cartilage, in that order. In one example, the nutrient fluid contacts adipose, cartilage, and synovium, in that order.

The tissue media can be common culture media formulations, such as Dulbecco's Modified Eagle's Medium (DMEM), which is readily available from commercial sources. The media may include serum.

Serum supplemented medium generally refers to supplementation with serum, such as fetal bovine serum, commonly at 10% (v:v). A “high serum” concentration can be a concentration of at least 10%, such as 10%, 11%, 12%, 13%, 14%, 15%, 16%, 17%, 18%, 19% or 20%. A “low serum concentration” can be 2% or lower, such as 1.5%, 1%, 0.5% or 0-%. “Low serum” medium includes “serum free” medium. An example of a high-serum universal medium that can be used is: DMEM, high glucose, pyruvate (Gibco), containing 10% fetal bovine serum (FBS, Invitrogen), 1X Antibiotic-Antimycotic (Gibco). An example of a low serum (in this case serum-free) medium: DMEM, high glucose, pyruvate (Gibco), 1X antibiotics-antimycotic (Gibco), and 1X Insulin-Transferrin-Selenium. The serum need not be fetal bovine serum. Other examples include human serum or serum from other mammals.

In the native joint, the articular cartilage is bathed in serum-free synovial fluid on one side and connected on the other side to subchondral bone that is vascularized. The oxygen tension of synovial fluid in humans is reported to be 6.5-9.0%. These conditions can be modeled with the neu-microJoint provided herein.

Normoxic fluid has no adjustment of oxygen tension and can therefore retain an atmospheric level of approximately 20% oxygen. Hypoxic medium is generated and maintained, for example, by including an inert gas such as nitrogen, in a gas supply which renders a lower oxygen tension of 5-8% to simulate known oxygen tension in the articular joint of about 6-7%. Hypoxic medium can be generated, for example, by using a hypoxic chamber to generate hypoxic medium, which is then perfused through the bioreactor. For example, such a chamber is available from Coy Laboratory Products (Grass Lake, MI).

### Methods

Provided herein are methods of using the disclosed neu-microJoint bioreactor to reproduce the biological conditions (e.g., pain) in a mammalian joint, and assess pharmaceutical agents for modification of the biological condition. Thus, one aspect of the neu-microJoint bioreactor described herein is its ability to mimic the tissue relationships within the osteochondral complex of the articular joint and to characterize responses to mechanical, toxicological, pathological and inflammatory insults or perturbations.

The application of the devices, systems, and methods described herein toward these types of studies typically proceeds according to several steps.

First, behavior of the tissue grown using the neu-microJoint bioreactor can be assessed under non-stressed conditions to confirm proper matrix production, differentiation marker expression, and baseline neuronal activity. Second, the system can be perturbed with mechanical, chemical, and/or toxicological stresses, insults, or other perturbation to demonstrate that the tissue responds according to published in vivo studies. Third, once validated, the system can be used to investigate biological process. For example, to study the effects of mechanical injury, the cartilage component can be pre-injured prior to tissue assembly to study the effects of damaged cartilage on neuronal activity observed in the neu-microJoint bioreactor. Alternatively, the assembled and matured tissue can be impacted and corresponding neuronal activity observed. Similarly, the tissue system can be employed as a high-throughput in-vitro model to assess the effects of treatment with glucocorticoids, pro-inflammatory cytokines, anti-inflammatory biologics, even biomaterial wear debris, such as titanium and polyethylene microparticles, on osteochondral health, etc. Systems grown using the devices, systems, and methods described herein offer novel capabilities for investigating the pathogenic mechanisms of OA as well as serving as a high-throughput platform to test candidate DMOADs.

Neural activity can be measured in the central chamber where nerves are cultured, or in the innervated outer chambers, using the electrodes (e.g., MEAs) embedded in these chambers. Additionally, Ca²⁺ imaging of the sensory neurons can be used to measure neural activity.

Thus, the methods can include introducing a preselected biological perturbation into at least one of the chambers in the device, and measuring neuronal activity in the central chamber before, during and after introduction of the preselected biological perturbation. The preselected perturbation can include one or more of a chemical perturbation, a toxicological perturbation, a mechanical perturbation, a physical perturbation, a biological perturbation, a disease initiator, an active agent, a chemical compound, a hormone, an inflammatory agent, a disease-modifying agent or a therapeutic agent.

For example, the tissue can be mechanically injured by providing a pathogenic load, and the response measured. In one example, bone pathology can be investigated by treating an osteoblast construct with glucocorticoids and measuring the response. In another example, bone inflammation can be investigated by treating an osteoblast construct with pro-inflammatory cytokines (e.g., TNF-α, etc.) and measuring the response. In yet another example, bone exposure to particulates can be investigated by treating an osteoblast construct with titanium microparticles and measuring the response. In another example, the tissues can be exposed to any of various implant wear debris, such as microparticles of ultra-high-molecular-weight polyethylene (UHMWPE), titanium, chromium/cobalt, etc., and the response measured. In another example, the tissues can be exposed to various cells, such as cells typical of an inflammatory environment, and the response measured. In each of these examples, the response can be measured using, e.g., ELISA, imaging. histology/IHC, mCT, MRI, or matrix metalloproteinases (“MMP”) activity techniques of cells in the outer chambers, as well as by monitoring electrical activity of the sensory neurons in the central chamber.

In some examples, a mechanical loading system can be provided that is configured to provide a physiological load to the tissue in the bioreactor. Such a loading system can then be verified using, e.g., mCT, MRI, histology/IHC, or imaging techniques.

In some examples, mechanical actuation or perturbation of tissues in a bioreactor, as described herein, can comprise a “gentle” application of load, for instance <10% strain for 1 hour a day, that mimics the general mechanical environment of the joints without causing damage, and it generally promotes the production and maintenance of better tissue. In other examples, mechanical actuation or perturbation can comprise >10% strain that can induce a response similar to an injury response.

In one exemplary design, the loading device includes a 3 mm loading surface having an unloaded position <0.5 mm from the cartilage surface, and is configured for loading of 5% strain (100 μm) at 0.1 Hz. This combination of strain and loading rate should be chondro-stimulatory in engineered cartilage constructs. Furthermore, extreme loading can be applied in conjunction with stimulation by biochemical stresses to simulate physical injury within the microtissue system. In alternative examples, the mechanical loading can be force- or stress-driven rather than strain-driven.

In another example, IL1β is used to trigger OA-like conditions in the outer chambers of the neu-microJoint bioreactor. For example, 10 ng/mL IL1β is introduced into the “synovial” flow for about 1 week. Neuronal activity can be assessed before, during and after induction of the OA-like conditions. These assays can be replicated with and without the presence of control and test agents for blocking the neural signaling (e.g. pain signaling) induced by OA.

### III. Additional Embodiments

Clause 1. A bioreactor comprising:
i) a first chamber comprising an upper part and a lower part, wherein the upper part of the first chamber comprises a first tissue comprising osteoblasts within a first scaffold, and the lower part of the first chamber comprises a second tissue comprising chondrocytes within a second scaffold;
ii) a second chamber comprising an upper part and a lower part, each comprising synovial cells within a third scaffold;
iii) a third chamber comprising an upper part and a lower part, each comprising fat pad cells within a fourth scaffold;
iv) a fourth chamber comprising neurons and the fourth chamber is interconnected to at least one of the first, second, or third chambers by microchannels configured to allow growth of neurites of the neurons from the fourth chamber to the at least one of the first, second, or third chambers;
v) a first influx conduit that supplies a first nutrient fluid to the upper part of the first chamber, and a first efflux conduit that removes the first nutrient fluid from the upper part of the first chamber;
vi) a second influx conduit that supplies a second nutrient fluid to the lower part of the first, second, and third chambers, and a second efflux conduit that removes the second nutrient fluid from the lower part of the first, second, and third chambers;
vii) a third influx conduit that supplies a third nutrient fluid to the upper part of the second chamber, and a third efflux conduit that removes the third nutrient fluid from the upper part of the second chamber; and
viii) a fourth influx conduit that supplies a fourth nutrient fluid to the upper part of the third chamber, and a fourth efflux conduit that removes of the fourth nutrient fluid from the upper part of the third chamber;
viii) a fifth influx conduit that supplies a fifth nutrient fluid to the fourth chamber, and a fifth efflux conduit that removes the fifth nutrient fluid from the fourth chamber; and wherein:
the lower parts of the first, second, and third chambers are interconnected;
the second nutrient fluid maintains separation from the first, third, and fourth nutrient fluids through functional contact between the upper and lower parts of the first, second and third chambers;
the first tissue is exposed to the first nutrient fluid and not the second, third, or fourth nutrient fluids;
the second tissue is exposed to the second nutrient fluid and not the first, third, and fourth nutrient fluids;
the first and second tissues remain in direct contact with each other; and
the bioreactor comprises a perturbation source that provides a preselected perturbation to at least one of the first, second, third, or fourth chambers of the bioreactor.

Clause 2. The bioreactor of clause 1, wherein the fourth chamber is interconnected to the first, second, and third chambers by microchannels configured to allow growth of neurites of the neurons from the fourth chamber to the first, second, and third chambers.

Clause 3. The bioreactor of clause 1 or clause 2, wherein the fourth chamber is interconnected to the first chamber, and the bioreactor further comprises a fifth chamber comprising neurons and interconnected to the second chamber, and a sixth chamber comprising neurons and interconnected to the third chamber, wherein the chambers are interconnected by microchannels configured to allow growth of neurites of the neurons of the fourth, fifth, and sixth chambers to the first, second, and third chambers, respectively.

Clause 4. The bioreactor of any one of the prior clauses. wherein the fourth, fifth, and/or sixth chambers further comprise one or more electrodes for stimulating or recording a neural signal from the neurons in the fourth, fifth, and/or sixth chambers.

Clause 5. The bioreactor of clause 4, wherein the electrodes are electrodes of one or more microelectrode arrays present in the fourth, fifth, and/or sixth chambers.

Clause 6. The bioreactor of any one of the prior clauses, wherein the osteoblasts, chondrocytes, synovial cells and/or fat pad cells are produced from mesenchymal stem cells or induced pluripotent stem cells within the bioreactor.

Clause 7. The bioreactor of any one of the prior clauses, wherein there is an additional tissue layer comprising mesenchymal stem cells or a semi-permeable membrane between the osteoblasts and the chondrocytes in the first chamber.

Clause 8. The bioreactor of any one of the prior clauses, wherein the first, second, third, and fourth chambers are all interconnected.

Clause 9. The bioreactor of any one of the prior clauses, wherein the preselected perturbation is one or more of a chemical perturbation, a toxicological perturbation, a mechanical perturbation, a physical perturbation, a biological perturbation, a disease initiator, an active agent, a chemical compound, a hormone, an inflammatory agent, a disease-modifying agent or a therapeutic agent.

Clause 10. The bioreactor of clause 9, wherein the disease modifying agent is one or more of an anti-osteoarthritic agent, an anti-diabetic agent, a cartilage anabolic or catabolic gene sequence, a bone anabolic or catabolic gene sequence, a macrophage stimulator, or a macrophage inhibitor.

Clause 11. The bioreactor of any one of the prior clauses, wherein the osteoblasts, chondrocytes, synovial cells and/or fat pad cells are from the same subject or stem cell.

Clause 12. The bioreactor of clause 11, wherein the subject is a mammal with a disease, and wherein the disease is one or more of osteoarthritis, a diabetes-associated joint complication, osteosarcoma, or a bone tumor.

Clause 13. The bioreactor of any one of the prior clauses, wherein the fourth chamber is interconnected to at least one of the first, second, or third chambers by microfluidic conduits.

Clause 14. The bioreactor of clause 13 wherein the microfluidic conduits transport fluids by diffusion or perfusion.

Clause 15. The bioreactor of any one of the prior clauses, wherein the first, second, and third chambers are interconnected, such that the second nutrient fluid entering through the second influx conduit contacts the chondrocytes in the first chamber, the synovial cells in the second chamber, and the fat pad cells in the third chamber.

Clause 16. The bioreactor of any one of the prior clauses, wherein the first nutrient fluid is normoxic; and
wherein the second nutrient fluid is hypoxic.

Clause 17. The bioreactor of clause 16, wherein the third nutrient fluid is normoxic and comprises 10% to 20% serum.

Clause 18. The bioreactor of clause 17, wherein the fourth nutrient fluid is normoxic and comprises 10% to 20% serum.

Clause 19. A method of reproducing the biological conditions in a mammalian joint, comprising:
circulating, in the bioreactor of any one of the prior clauses, the first nutrient fluid through the upper part of the first chamber of the bioreactor, circulating the second nutrient fluid through the lower parts of the first, second, and third chambers of the bioreactor, and circulating the fifth nutrient fluid through the fourth, fifth, and/or sixth chambers of the bioreactor;
thereby reproducing the biological conditions in a mammalian joint.

Clause 20. The method of clause 19, further comprising introducing the preselected perturbation into at least one of the first, second, or third chambers of the bioreactor.

Clause 21. The method of clause 20, wherein the preselected perturbation comprises one or more of a chemical perturbation, a toxicological perturbation, a mechanical perturbation, a physical perturbation, a biological perturbation, a disease initiator, an active agent, a chemical compound, a hormone, an inflammatory agent, a disease-modifying agent or a therapeutic agent.

Clause 22. A method of reproducing the biological conditions in a mammalian joint, comprising:
circulating, in the bioreactor of any one of the prior clauses, the first nutrient fluid through the upper part of the first chamber of the bioreactor comprising the osteoblasts;
circulating the third nutrient fluid through the upper part of the second chamber of the bioreactor comprising the synovial cells;
circulating the fourth nutrient fluid through the upper part of the third chamber of the bioreactor comprising the fat pad cells;
circulating the fifth nutrient fluid through the fourth, fifth, and/or sixth chambers of the bioreactor
circulating the second nutrient fluid through the lower parts of the first, second, and third chambers of the bioreactor; wherein the second nutrient fluid contacts the chondrocytes in the lower part of the first chamber, the synovial cells in the lower part of the second chamber, and the fat pad cells in the lower part of the third chamber;
thereby reproducing the biological conditions in a mammalian joint.

Clause 23. The method of clause 22, further comprising introducing the preselected perturbation into at least one of the first, second, or third chambers of the bioreactor.

Clause 24. The method of clause 23, wherein the preselected perturbation comprises one or more of a chemical perturbation, a toxicological perturbation, a mechanical perturbation, a physical perturbation, a biological perturbation, a disease initiator, an active agent, a chemical compound, a hormone, an inflammatory agent, a disease-modifying agent or a therapeutic agent.

### EXAMPLES

The following examples are provided to illustrate particular features of certain aspects of the disclosure, but the scope of the claims should not be limited to those features exemplified.

### Example 1

### Neu-MicroJoint Bioreactor

This example illustrates an exemplary three-dimensional, multi-component microphysiological joint chip referred to as an innervated microJoint (neu-microJoint) bioreactor, and which can be used, for example, to study mechanisms of osteoarthritis-associated pain, develop novel treatments, screen novel treatments, and identify effective treatment options for individual patients (personalized pain medicine).

FIGS. 1-10 depict the neu-microJoint bioreactor, in which bone, cartilage, synovium, adipose, and neuronal tissues are integrated. Constructs of bone, cartilage, synovium, and adipose tissues are located in the outer chambers. In the center of the device is a smaller chamber, which is used to house sensory neural cells. These neural cells innervate the tissue constructs in the outer chambers via microfluidic channels. The microfluidic channels provide surface tension and fluid resistance to enable a physical connection between the neural chamber and the outer chambers in the neu-microJoint bioreactor, while restricting bulk fluid exchange. Thus, it is possible to maintain the phenotype of each tissue type with tissue specific culture media, while enabling innervation of the tissue. The neural cells can be cultured in 2D, on a glass surface that forms the floor of the central and outer chambers, enabling monitoring of neural activity using standard imaging and electrophysiological methods.

The outer chambers are each filled with a chamber insert that contains tissue construct(s) and separates the individual chambers into upper and lower parts. The upper and lower parts of each of the outer chambers are connected to fluid conduits in the shell that provide separate fluid flows to the lower parts of each outer chamber, and a single flow path linking the upper parts of the outer chambers. The upper and lower parts of the outer chambers are separated by a barrier located in the chamber body insert and the seal between the chamber body insert and the wall of the outer chamber.

In this example, the upper portion of the neu-microJoint bioreactor is 3D printed from biocompatible resin, which is then bonded to a PDMS base. The 3D-printed top portion contains the influx and efflux conduits, and hollow cylinders for the four tissue chambers. To generate the part with 3D printing, a 3D model was first created in Solidworks software, and then converted into an STL file. Using the STL file as the template, the part will be printed by a 3D printer from EnvisionTec. The PDMS base contains matching hollow cylinders for the four tissue chambers, and is bonded to the top portion such that the tissue chambers extend through the top portion and the base. The base is created by casting PDMS on a special wafer, which allows the generation of microchannels on one side of PDMS. The void chambers are created by coring out the PDMS with a sharp punch. See FIG. 5. In this example the microchannels are 3-5 μm in height. The bottom of the neu-microJoint bioreactor, and the floor of the four chambers is provide by a glass layer that is plasma bonded to the PDMS base. Because any clean and flat glass surface will do for the base of the neu-microJoint bioreactor, the glass surface can be a coverslip to facilitate live cell imaging or a microelectrode array to enable simultaneous imaging and electrical recording of neural activity.

The perfusion ports are tapped and sterile tubing connectors attached. Thus, the microfluidic component of the neu-microJoint bioreactor is made of PDMS, a biocompatible material that can be poured over a mask and light cured. PDMS can also be plasma bonded to glass, preventing leaks and establishing the floor of the microfluidic channel. A bio-adhesive is then used to attach the 3D printed part of the bioreactor to the PDMS base.

The joint tissues were generating by seeding human stem cells into a gelatin-based hydrogel within the chamber body inserts, which are also pre-created using 3D printing. Along with the o-ring, the inserts create a barrier to separate the top and bottom parts of the tissue chambers, and the tissue medium flows through these chambers. After tissues are mature (around 4 weeks), DRG neurons are seeded into the central chamber, which then innervate tissues through the microchannels.

The fluid flow through the lower part of each of the outer chambers is independent, as indicated by the arrows of FIG. 2C. Thus, the nutrient fluids perfusing the tissue constructs in the lower parts of each chamber can be optimized for the particular tissue type in the chamber. To model the synovial fluid that baths all components of the joint in vivo, the neu-microJoint bioreactor also contains a common perfusion element to enable cross-talk between the upper parts of the outer chambers of the device, illustrated by the grey arrows in FIG. 2C. This perfusion element helps to model OA pathogenesis.

In vivo, the cell bodies of sensory neurons that innervate joint tissues reside in dorsal root ganglia (DRG), located adjacent to the spinal cord and consequently a considerable distance from the joint. This anatomy is modeled in the neu-microJoint bioreactor, with the sensory neuron cell bodies maintained in isolation from the joint tissues. The only connections between the neurons and the tissues are provided by the neurites. Consequently, activity detected in the cell body is evoked by mediators released from the tissues that are able to generate action potentials. The neurites also physically connect with joint tissues, which allows studying the direct contribution of mechanics on neurites within tissues.

This 2D (neural culture)/3D (joint tissues) hybrid system enables assessment of changes in neural activity with imaging and neural recording coupled with innervation of tissue grown in 3D. 3D tissues permit more accurate representation of tissue function and phenotype, as well as the ability to apply mechanical (and therefore more physiologically relevant) stimuli. Separating the tissues, but allowing cross-talk and independent innervation, means that it is possible to assess the tissue(s) responsible for the pain signaling of OA, whether there are tissues that release compounds that attenuate the pain of OA, and how the response of any one tissue type influences the phenotype of the others (and is therefore a primary or secondary driver for OA pain).

For the sensory neurons in the central chamber, human primary sensory neurons or induced pluripotent stem cells (iPSC)-derived sensory neuron progenitors (iNeuron) were cultured in the bioreactor chamber, in a standard 2D culture. Human DRG neurons can be recovered from organ donors and cultured. In addition to human primary sensory neurons, non-human sensory neurons as well as iPSC-derived neurons may also be used. iPSCs can be generated from a patient's own cells, enabling investigation of genetic differences that may contribute to increased pain in subpopulations of OA patients thereby facilitating the development of personalized treatments.

Acutely dissociated DRG neurons are plated in the neural chamber and cultured in 2D at a density of ˜10 neurons/100 μm² to enable recording of neural activity, for example with microelectrodes or MEAs embedded in the floor of the chamber.

The sensory neurons are cultured in the neu-microJoint bioreactor for a period of time sufficient for neurites to grow through the microchannels and into the outer chambers. The neurons maintain a high viability (>80%) for at least four weeks. Neural processes coursing through the microchannels spanning a distance of >8 mm are detectable within 7 days of plating. Once the neurites innervate the outer tissue chambers, the processes extend over as well as into the gelatin scaffolds. Neurite extension into the tissue chambers is assessed histologically where neural processes are easily visualized with antibodies against PGP9.5 or βIII-tubulin.

For the tissue scaffolds containing synovial cells, adipose cells, chondrocytes, or osteocytes, human mesenchymal stem cells (MSCs, >98% positive to CD73, 90, 105 and >99% negative to CD31, 34, 45, capable of colony formation and trilineage differentiation) or human iPSCs-derived MSCs (iMPCs) are encapsulated in photo-crosslinked gelatin scaffolds, and then subjected to tissue specific media to generate individual synovium, adipose, osteochondral tissues, as well as an additional individual macrophage component. After maturation, these tissue constructs are assembled with the chamber body inserts as described herein and inserted into the outer chambers of the neu-microJoint bioreactor, the fluid conduits are tapped and connected to suitable tubing, and tissue culture medium is pumped through the device to perfuse the tissue scaffolds. To maintain the respective phenotype of each component, but also allow the tissue crosstalk, the lower part each tissue chamber is perfused with tissue specific media and the upper part is perfused with a fluid common to all chambers.

To enable recording of neural activity, sensory neurons are plated on a high-density microelectrode array (MEA) embedded in the floor of the neuron bioreactor chamber. Additionally, over the course of 28 days, the neurites reach the synovium, bone, and adipose chambers. In addition, they are functional, as assessed by the ability to maintain physiological cytosolic Ca²⁺ concentrations (˜100 nM), a healthy resting membrane potential <−50 mV, and overshooting action potentials in response to physiological stimuli.

One way to distinguish neurons innervating tissue chambers from those that have not is to dope the tissue chambers with retrograde tracers such as quantum dots. The use of different colored dots in each chamber enables determination of the chambers innervated as well as whether a neuron had innervated more than one chamber. Adding micro electrode arrays to the tissue compartments enable the recording of efferent activity in the tissue chambers, as well as confirm that it was possible to detect orthodromically conducted action potentials in the neuron chamber. Changes in intracellular Ca²⁺ can also be used to measure activity in neurons.

DRG neurons cultured on MEAs for 4 days in the central chamber of the neu-microJoint bioreactor were screened with increasing concentrations of KCl (10, 30, and 50 mM) to detect spiking. There was little to no spontaneous activity under baseline conditions, but activity was detected in response to 30 mM KCl.

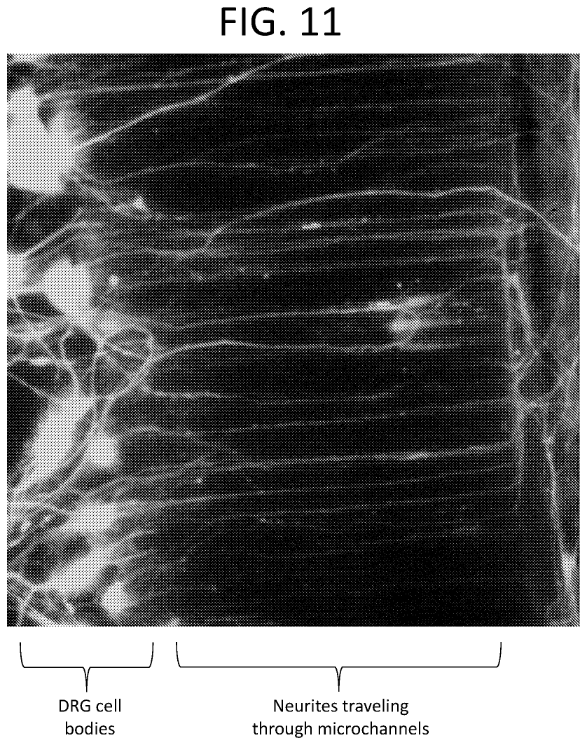

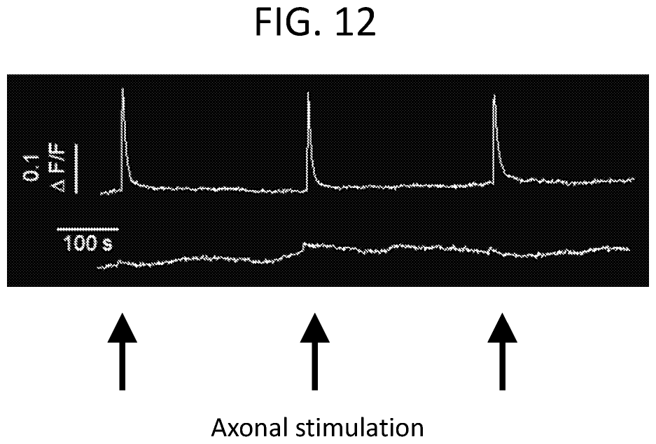

Rodent and human DRG neurons plated in the neu-microJoint cultured between 10-28 days showed substantial axonal growth through the microchannels as visualized through DiI or fura2 labeling (FIG. 11). Moreover, electrical stimulation applied to the neurites evoked an increase in calcium in the corresponding soma (FIG. 12).

Conditioned media from the OA-modeled microJoint applied to the neurons caused an increase in calcium in a subpopulation of neurons. These data show the potential to apply an in vitro microphysiological system to study OA pain.

### Example 3

### Modeling Joint Pain Using the Neu-Microjoint Bioreactor

This example illustrates methods of modeling OA pain with the neu-microJoint bioreactor. The influence of simulated OA-like conditions in the outer chambers on neural activity in the central chamber is studied.

In a first method of modeling OA pain with the neu-microJoint bioreactor, IL1β is used to trigger OA conditions in the outer chambers of the neu-microJoint bioreactor. To create the IL1β OA model using the neu-microJoint bioreactor, the device is constructed as described in Example 1, and 10 ng/mL IL1β is introduced into the “synovial” flow for 1 week. OA induction is confirmed on the basis of gene expression profiling, and biochemical and histological analyses (see Lin et al., Stem cell-based microphysiological osteochondral system to model tissue response to interleukin-1beta. Mol Pharm. 2014; 11 (7):2203-12). Tissue-specific anabolic and catabolic genes are tested. The “synovial” eluate will be collected 1 day after the withdrawal of IL1β.

IL1β challenged synovium caused significant increases in both MMP13 and a disintegrin and metalloproteinase with thrombospondin motifs (ADAMTS) 4 mRNA expression in cartilage (in the upper part of the bone/cartilage chamber), which was not directly exposed to the IL1β. Since IL1β that is used to generate the OA model is only applied to the flow of synovium, it indirectly stimulates the other tissues. However, it may directly sensitize nociceptive afferents innervating this chamber. As this may be part of the OA process, retrograde tracers may be used to distinguish activity in neurons innervating the synovium chamber from those innervating other chambers, and corresponding neural activity is monitored.

Natural bone and cartilage growth is known to be affected by mechanical stresses encountered by those tissues as they grow, thus systems allowing the introduction of such stresses can facilitate tissue growth which more accurately resembles native tissue growth. Thus, in a second method of modeling OA pain with the neu-microJoint bioreactor, a mechanical loading pattern downward on tissue/fluid is used to trigger OA conditions in the outer chambers of the neu-microJoint bioreactor. The outer chambers of the bioreactor are configured to receive a chamber body insert and a sealing lid containing a mechanical actuator or piston that applies downward pressure on the chamber, as described in U.S. Pat. No. 11,339,362

Macrophages can be added to any of the tissue scaffolds in the bioreactor to assess their contribution to joint pain modeled by the neu-microJoint bioreactor. M1 macrophages are a classically activated pro-inflammatory cell type that express pro-inflammatory cytokines, chemokines and many other substances. M2 macrophages are alternatively activated and have an anti-inflammatory pro-tissue regenerative phenotype. Consistent with the pro-inflammatory nature of M1 macrophages, PGE2, IL-6 and tumor necrosis factor (TNF)-α, all of which are factors generated by M1 macrophages, sensitize sensory neurons in fura-2 Ca²⁺ imaging and whole cell patch recordings.

Recording of electrical activity as well as calcium imaging in the sensory neurons is performed before, during, and after induction of OA like conditions in the outer chambers of the neu-microJoint bioreactor. Test or control agents for modification of joint pain can be introduced into the system for assessment.

Further, to quantify the impact of OA on neural sprouting, the number of fibers per unit area on the surface of each tissue compartment is quantified, along with the depth and density (again as a fiber number per unit area) into the tissue compartments. Tissue will be stained with a neural marker such as PGP9.5, or a neuron subtype specific marker such as CGRP (see Wimalawansa SJ. Calcitonin gene-related peptide and its receptors: molecular genetics, physiology, pathophysiology, and therapeutic potentials. Endocr Rev. 1996; 17 (5):533-85). To ensure the rigor and reproducibility of results, experimenters may be blinded as to whether OA had been induced in the Neu-microJoint, results will be replicated on neurons from at least three different preparations from males and females (humans), and Neu-microJoints with and without OA will be run in parallel. Because spontaneous pain is not an initial feature of OA, resting activity may not be detected. The presence of sensitization will be assessed by quantifying changes in the response to the focal application of depolarizing stimuli, like elevated K+.

Additionally, modeling of degradation of joint tissues, including increased release of carboxy-terminal telepeptides of type II collagen (CTX II), MMP-13, and pro-inflammatory cytokines, identified by real time PCR and ELISA may be measured. Tissue degradation should be associated with the activation and/or sensitization of nociceptors. An initial decrease in nociceptor excitability is possible, which would suggest the release of anti-inflammatory mediators that may be involved in the suppression of pain during the onset of OA.

### Example 4

### Assessment of Pharmacological Agents Using the Neu-MicroJoint Bioreactor

This example illustrates validation of the neu-microJoint with known joint pain-reducing agents, and its use to identify and test novel therapeutic agents for ameliorating joint pain.

To further validate the neu-microJoint as a model system with which to identify novel mechanisms mediating pain associated with joint injury as well as the efficacy of novel therapeutic interventions, the impact of agents with known clinical efficacy is assessed.

The sensitization of knee afferents is a primary driver for OA pain and hypersensitivity. Hyper-innervation of the joint may also contribute to OA pain, but it may also be a protective mechanism, facilitating regenerative processes. While a number of trophic factors such as NGF, cytokines such as TNF-α and IL-6, and inflammatory mediators such as prostaglandin E2 (PGE2) appear to be upregulated in models of OA, and all have been shown to sensitize nociceptive afferents, it remains to be determined which, if any of these are responsible for the pain of OA. Furthermore, it remains to be determined which tissue compartment(s) are the source of the mediators primarily responsible for OA pain. The possibilities include macrophages, bone, and the Infrapatellar Fat Pad (IPFP). On the other hand, there is evidence for injury-induced changes in sensory neurons, suggesting it is also possible that injury-induced changes in the joint drive changes in the sensory innervation that are ultimately responsible for the pain and hypersensitivity of OA.

The efficacy of drugs that reduce OA pain in the clinic against the “pain” associated with OA models is assessed in the Neu-microJoint. The drugs tested include ibuprofen and celecoxib (NSAIDs), Tanezumab (TAB-111, monoclonal antibody to NGF), and Mavatrep (TRPV1 antagonist). Drugs are tested on naïve joints modeled with the neu-microJoint, as well as in modeled OA joints, in which OA has been induced with IL1β, or mechanical stimulation. Resting and evoked neural activity is examined, before and after treatment with these pain medications, with or without the inclusion of adipose element in the Neu-microJoint.

Further, RNAseq and proteomic/metabolomic approaches are used to screen for changes in tissue compartments associated with the models of OA. By comparing changes in tissue compartments at the time with neural sensitization is first detected with a time when spontaneous activity is detected, factors responsible for both the initiation and maintenance of OA pain are identified. Results from this screen may reveal biomarkers predictive of the presence of ongoing pain as well as for the efficacy of therapeutic interventions. Pathway analysis may also reveal novel therapeutic targets for the treatment of OA pain. For example, changes in neuronal transporters and pumps may predict an increase in intracellular Cl—which could underlie an excitatory action of GABA released from immune cells. In this way the “omic” data, will be used to guide the exploration of novel therapeutic approaches for the treatment of OA pain. Ideally, the pathway analysis would suggest targets for which there are drugs already available that could be re-purposed for the treatment of OA, such diazepam, or other positive allosteric modulators of the GABA_{A} receptor.

The influence of a test drug on the time of onset of neural activity, as well as the magnitude of the resting and evoked response and the threshold for activation. OA is induced with IL1β or mechanical stimulation. Since OA drugs are utilized after the emergence of OA pain, drug treatment in all the assigned test chambers is initiated after the emergence of neural activity in the OA groups. Treatments include the following where initial concentrations were derived from published literature: (A) Ibuprofen (from Sigma, 10 mg/ml; (B) celecoxib (from Sigma, 10 μM; (C) TAB-111 (from Creative Biolabs, 10 ng/ml; (D) Troglitazone, a type of TZD, from Sigma, 1 μM. Additional 10-fold higher/lower concentrations (3-4 for each) are used to confirm effects are dose-dependent and/or that negative results are not due to incomplete target coverage. Changes in tissue phenotype (gene expression, histology), and neural activity and sprouting are characterized.

The impact of the OA models on changes in gene expression in the different tissue compartments, as well as the mediators produced and released. is assessed. Comparisons are made between expression/protein levels from naïve tissue/fluid in each compartment run in parallel with that following the induction of OA with IL1β or mechanical stimulation. Data is collected at two time points: the first detectable sign of sensitization (i.e., initiation of OA) and after the establishment of spontaneous activity (i.e., maintenance of OA). For RNAseq, tissue is collected at each time point and immediately placed in RNALater. Total RNA is isolated with RNEasy® kit from Qiagen, and DNA removed by digestion. PolyA+ RNA libraries will be prepared for sequencing with Illumina Truseq® RNA sample preparation kit. To quantify changes in relative transcript abundance, the sequenced FASTQ files for each sample are first mapped onto the human transcriptome (NCBI hg19 for genome and Gencode v14 for transcriptome). RNAseq® data sets are analyzed with Tophat/Cufflinks pipeline (Trapnell et al. Differential gene and transcript expression analysis of RNA-seq experiments with TopHat and Cufflinks. Nat Protoc. 2012; 7(3):562-78). Changes in relative abundance are quantified with the tool Cuffdiff (Trapnell C, Hendrickson D G, Sauvageau M, Goff L, Rinn J L, Pachter L. Differential analysis of gene regulation at transcript resolution with RNA-seq. Nat Biotechnol. 2013; 31(1):46), and calculated based on the number of fragments sequenced that map to a specific gene as the fragments per kilobase per million mapped fragments (FPKM). For proteomic/metabolomic data collection and analysis, we start with Mass Spectrometry (MS) based approaches. Protein is extracted from issue compartments with phosphatase inhibitors. Following centrifugation, lysates are collected, and protein concentration determined with BCA kits. Following protein digestion, fractionation and desalting, peptides are analyzed by MS. Similarly, 100 μl of fluid from each compartment may be processed for analysis via GC-TOF/MS. GC data may be analyzed with software used to align metabolites by spectral match and retention time, where internal standards are used to confirm alignment. Standardized data may be analyzed via principal component analysis with the SIMCA-P software package and orthogonal partial least squares discriminant analysis (OPLS-DA). The OPLS-DA model will be used to determine the variable importance for projection (VIP) value for each metabolite. Metabolites with a VIP>1, fold>1.5, and a p<0.05 will be considered different between naïve and OA samples. ELISA may be used to further validate and quantify changes in gene expression and protein levels identified with screening approaches.

Pathway analysis will be used to predict points of convergence between changes in gene expression and proteins detected. If points of convergence are not readily “druggable”, pathway analysis will also be used to predict up-stream and/or downstream targets that may be druggable. While we could use an unbiased screen for inhibitors of OA-induced neural sensitization and/or activation, we will start with targets predicted by our “omic” analysis. Ideally, it will be possible to test drugs already approved for use in patients that may be re-purposed as novel targets for the treatment of OA.

In view of the many possible examples to which the principles disclosed herein may be applied, it should be recognized that the illustrated examples are only preferred examples and should not be taken as limiting the scope of the disclosure. Rather, the scope of the disclosure is at least as broad as the following claims. We therefore claim all that comes within the scope and spirit of these claims.

1. A bioreactor, comprising:
i) a first chamber comprising an upper part and a lower part, wherein the upper part of the first chamber comprises a chondrocytes within a tissue scaffold, and the lower part of the first chamber comprises a osteoblasts within a tissue scaffold;
ii) a second chamber comprising an upper part and a lower part, each comprising synovial cells within a tissue scaffold;
iii) a third chamber comprising an upper part and a lower part, each comprising adipose cells within a tissue scaffold;
iv) a fourth chamber comprising sensory neurons in a two-dimensional culture, wherein the fourth chamber is interconnected to the lower parts of the first, second, and third chambers by microchannels;
v) a first influx conduit that supplies a first nutrient fluid to the lower part of the first chamber, and a first efflux conduit that removes the first nutrient fluid from the lower part of the first chamber;
vi) a second influx conduit that supplies a second nutrient fluid to the lower part of the second chamber, and a second efflux conduit that removes the second nutrient fluid from the lower part of the second chamber;
vii) a third influx conduit that supplies a third nutrient fluid to the lower part of the third chamber, and a third efflux conduit that removes the third nutrient fluid from the lower part of the third chamber;
viii) a fourth influx conduit that supplies a fourth nutrient fluid to the upper parts of the first, second, and third chambers, and a fourth efflux conduit that removes the fourth nutrient fluid from the upper parts of the first, second, and third chambers,
and wherein:
the microchannels are configured to allow growth of neurites of the sensory neurons from the fourth chamber to the lower parts of the first, second, and third chambers, and to limit bulk flow of the first, second, third, and fourth nutrient fluids into the fourth chamber;
the upper parts of the first, second, and third chambers are interconnected by fluid conduits in series;
the upper and lower parts of the first, second and third chambers are separated by a barrier layer that permits biochemical communication but not cell migration between the upper and lower parts of the first, second and third chambers, respectively;
the chondrocyte tissue scaffold is exposed to the fourth nutrient fluid and not the first, second, or third nutrient fluids;
the osteoblast tissue scaffold is exposed to the first nutrient fluid and not the second, third, or fourth nutrient fluids;
the bioreactor comprises a perturbation source that provides a preselected perturbation to at least one of the first, second, third, or fourth chambers.

2. The bioreactor of claim 1, wherein the upper parts of the first, second, and third chambers are interconnected by fluid conduits in series, and the fourth influx conduit supplies the fourth nutrient fluid to the upper part of the chamber at one end of the series, and the fourth efflux conduit removes the fourth nutrient fluid from the upper part of the chamber at the other end of the series.

3. The bioreactor of claim 1, further comprising a fifth influx conduit that supplies a fifth nutrient fluid to the fourth chamber, and a fifth efflux conduit that removes the fifth nutrient fluid from the fourth chamber.

4. The bioreactor of claim 1, wherein
the osteoblast tissue scaffold in the lower part of the first chamber further comprises osteoclasts and/or endothelial cells;
the synovial tissue scaffold in the upper part of the second chamber comprises fibroblasts;
the synovial tissue scaffold in the lower part of the second chamber comprises fibroblasts and further comprises immune cells and/or endothelial cells;
the adipose tissue scaffold in the lower part of the third chamber further comprises immune cells and/or endothelial cells; and/or
the sensory neurons in the fourth chamber are incubated in the presence of immune cells.

5. The bioreactor of claim 1, wherein
the synovial scaffolds and/or the adipose scaffolds comprise non-polarized (M0) macrophages or macrophages polarized to M1 or M2 phenotype; and/or
the sensory neurons are incubated in the presence of non-polarized (M0) macrophages or macrophages polarized to M1 or M2 phenotype.

6. The bioreactor of claim 1, wherein the barrier layer is a heterologous tissue scaffold comprising mesenchymal stem cells or a semi-permeable membrane.

7. The bioreactor of claim 1, comprising a shell comprising a base forming the bottom of the first, second, third, and fourth chambers.

8. The bioreactor of claim 7, wherein the microchannels are adjacent to the base.

9. The bioreactor of claim 7, wherein the base is made of glass and is suitable for microscopic imaging of the sensory neurons in the fourth chamber.

10. The bioreactor of claim 1, wherein there are from 5 to 100 microchannels interconnecting the fourth chamber with each of the first, second, and third chambers.

11. The bioreactor of claim 1, wherein the microchannels are about 50 μm-about 2000 μm in length.

12. The bioreactor of claim 1, wherein the microchannels have a cross-sectional area of about 20 μm² to about 100 μm².

13. The bioreactor of claim 1, wherein the fourth chamber further comprises one or more electrodes for stimulating or recording neural signals from the sensory neurons.

14. The bioreactor of claim 13, wherein the electrodes are contained in a microelectrode array.

15. The bioreactor of claim 1, further comprising electrodes adjacent to the microchannels to induce orthodromic signals in neurites extending through the microchannels from the fourth chamber into the first, second, or third chamber.

16. The bioreactor of claim 1, wherein the osteoblasts, chondrocytes, synovial cells, fat pad cells, and/or sensory neurons are produced from mesenchymal stem cells or induced pluripotent stem cells within the bioreactor.

17. The bioreactor of claim 1, wherein the sensory neurons are dorsal root ganglion cells.

18. The bioreactor of claim 1, wherein the preselected perturbation is one or more of a chemical perturbation, a toxicological perturbation, a mechanical perturbation, a physical perturbation, a biological perturbation, a disease initiator, an active agent, a chemical compound, a hormone, an inflammatory agent, a disease-modifying agent or a therapeutic agent.

19. The bioreactor of claim 18, wherein the disease modifying agent is one or more of an anti-osteoarthritic agent, an anti-diabetic agent, a cartilage anabolic or catabolic gene sequence, a bone anabolic or catabolic gene sequence, a macrophage stimulator, or a macrophage inhibitor.

20. The bioreactor of claim 1, wherein the osteoblasts, chondrocytes, synovial cells and/or fat pad cells are from the same subject or stem cell.

21. The bioreactor of claim 20, wherein the subject is a mammal with a disease, and wherein the disease is one or more of osteoarthritis, a diabetes-associated joint complication, osteosarcoma, or a bone tumor.

22. The bioreactor of claim 1, wherein the nutrient fluids are normoxic or hypoxic.

23. A method of reproducing the biological conditions in a mammalian joint, comprising:
providing the bioreactor of claim 1, wherein the sensory neurons in the fourth chamber have neurites extending to the cells of the first, second, and third chambers;
circulating the first nutrient fluid through the lower part of the first chamber of the bioreactor comprising the osteoblasts;
circulating the second nutrient fluid through the lower part of the second chamber of the bioreactor comprising the synovial cells;
circulating the third nutrient fluid through the lower part of the third chamber of the bioreactor comprising the fat pad cells;
circulating the fourth nutrient fluid through the upper parts of the first, second, and third chambers of the bioreactor; wherein the fourth nutrient fluid contacts the chondrocytes in the upper part of the first chamber, the synovial cells in the upper part of the second chamber, and the fat pad cells in the upper part of the third chamber;
thereby reproducing the biological conditions in a mammalian joint.

24. The method of claim 23, wherein the bioreactor comprises a fifth influx conduit that supplies a fifth nutrient fluid to the fourth chamber, and a fifth efflux conduit that removes the fifth nutrient fluid from the fourth chamber, further comprising circulating the fifth nutrient fluid through the fourth chamber of the bioreactor comprising the sensory neurons;

25. The method of claim 23, further comprising introducing a preselected perturbation into at least one of the first, second, or third chambers of the bioreactor.

26. The method of claim 25, wherein the preselected perturbation comprises one or more of a chemical perturbation, a toxicological perturbation, a mechanical perturbation, a physical perturbation, a biological perturbation, a disease initiator, an active agent, a chemical compound, a hormone, an inflammatory agent, a disease-modifying agent or a therapeutic agent.

27. The method of claim 23, further comprising measuring neuronal activity in the fourth chamber.

28. The method of claim 27, wherein measuring neuronal stimulation comprises measuring action potentials with one or more electrodes or measuring intracellular Ca²⁺.

29. The method of claim 27, wherein measuring neuronal activity in the fourth chamber models pain sensation in the knee.
